(19) United States
(12) Patent Application Publication
(10) Pub. No.: US 2024/0358773 A1
(43) Pub. Date: Oct. 31, 2024

(54) Parabacteroides Goldsteinii to Treat or Prevent Inflammatory and AutoImmune Diseases

(71) Applicant: Joslin Diabetes Center, Boston, MA (US)

(72) Inventors: Aleksandar Kostic, Boston, MA (US); Lidan Zhao, Beijing (CN); Md Zohorul Islam, Sommerville, MA (US); Loc Duyen Pham, Quincy, MA (US); Tao Xu, Brighton, MA (US)

(73) Assignee: Joslin Diabetes Center, Boston, MA (US)

(21) Appl. No.: 18/640,699

(22) Filed: Apr. 19, 2024

Related U.S. Application Data
(60) Provisional application No. 63/460,768, filed on Apr. 20, 2023.

(51) Int. Cl. A61K 35/741; A61P 37/02

(52) U.S. Cl. CPC A61K 35/741; A61P 37/02

(57) ABSTRACT

This disclosure relates to compositions produced from Parabacteroides goldsteintii and their use to treat or prevent inflammatory and autoimmune diseases. This disclosure also relates to treatment or prevention of inflammatory and autoimmune diseases by administering an A2a adenosine receptor agonist.

### DESCRIPTION

This application claims priority to U.S. Provisional Application No. 63/460,768 filed Apr. 20, 2023, which is incorporated herein in its entirety for any purpose.

This invention was made with government support under Research Grant No. P30 DK036836 awarded by NIH/National Institute of Diabetes and Digestive and Kidney Diseases Diabetes. The government has certain rights in the invention.

### FIELD

This disclosure relates to compositions produced from Parabacteroides goldsteinii and their use to treat or prevent inflammatory and autoimmune diseases. This disclosure also relates to treatment or prevention of inflammatory and autoimmune diseases by administering an A2a adenosine receptor agonist.

### BACKGROUND

The importance of the gut microbiota to homeostatic immune function has been demonstrated by the wide range of immune functional and structural deficiencies observed in germ-free (GF) or antibiotic-treated mice (2-4) that can be rescued by microbial recolonization of the gut. Gut microbiota alterations caused by genetic susceptibility, antibiotic use, or Westernized diet, for example, can influence intestinal diseases, immune disorders, cancer, and the metabolic syndrome (6). However, it is necessary to understand the underlying molecular mechanisms to fully exploit the microbiota for therapeutic intervention. Currently, our knowledge of microbial immunomodulation is mainly mechanistically limited to activation of Toll-like receptors (e.g., TLR2/4/6), members of G protein-coupled receptors (GPCR 41/43/109a, P2XR, and TGR5), and nuclear receptors (AhR, PXR, FXR), or inhibition of histone deacetylase in immune cells, by microbially derived polysaccharides, short-chain fatty acids, and bile acids, and amino acid derivatives (7).

Previous studies collectively demonstrated the pleiotropic functions of the Altered Schaedler Flora (ASF) in resolving immunological, neurological, and metabolic defects in germ-free mice (Collins et al. 2014; Ivanov et al. 2008; Geuking et al. 2011; Mosconi et al. 2013; Hapfelmeier et al. 2010; Bouskra et al. 2008; Keilbaugh et al. 2005; Lavoie et al. 2019; Li et al. 2016). Some ASF members are presumably responsible for the rescued immunophenotypes, such as IL-10-dependent Treg proliferation in the colon (Geuking et al. 2011), but their identity is yet unknown.

In the present study, functionally competent ASF was used as a model of gut microbiota to dissect the role of gut microbes in immune regulation. Overall, intestinal Parabacteroides goldsteinii isolates were identified as potent immune tolerance promoting microbes through the production of extracellular vesicles (EVs), to regulate cytokine production, immune cell polarization and proliferation. Oral delivery of these gut microbes and purified EVs plays a protective role in mouse models of human inflammatory and autoimmune diseases, such as dextran sulfate sodium-induced colitis, collagen-induced arthritis, and type 1 diabetes. Since P. goldsteinii has a high prevalence and abundance in human feces, it can serve as a novel mechanism driven therapeutic avenue to counteract aberrant immunity.

The gut microbiota is critical to immune homeostasis (1-4), but understanding of the underlying molecular mechanisms is limited. Here, distinct immunophenotypes were promoted by members of the eight-membered “model microbiome” altered Schaedler flora (5). Parabacteroides goldsteinii ASF519 produces extracellular vesicles (EVs) that induce immune tolerance by promoting interleukin (IL)-10 production in myeloid cells via the activation of adenosine receptor A2a. Enhanced autophagy by EV treatment in dendritic cells contributes to IL-10 induction partly through the pannexin1-CD73-A2a axis. ASF519 colonization in mice increases the level of adenosine in ceca and induces IL-10 secreting dendritic cells in colonic lamina propria. These tolerogenic immunophenotypes were pharmacologically reversed by A2a blockage and reproduced by intraperitoneal injection of purified EVs. In mouse models of human autoimmune diseases, ASF519 supplementation significantly ameliorated insulitis in type 1 diabetes and collagen-induced arthritis. This study unveils a paradigm of gut microbiota-adenosine receptor interactions in immune tolerance mediated by EVs, and it potentially provides a new therapeutic strategy for immune disorders.

### SUMMARY

In accordance with the description, the present disclosure describes extracellular vesicles prepared from Parabacteroides goldsteinii and their administration to cells. The disclosure also describes prevention or treatment of autoimmune or inflammatory diseases by administration of (1) live Parabacteroides goldsteinii and/or a composition comprising isolated extracellular vesicles prepared from a sample comprising Parabacteroides goldsteinii, (2) an A2a adenosine receptor agonist, or (3) a composition comprising live Parabacteroides goldsteinii and/or a composition comprising isolated extracellular vesicles prepared from a sample comprising Parabacteroides goldsteinii together with an A2a adenosine receptor agonist.

Embodiment 1. A composition comprising isolated extracellular vesicles prepared from a sample comprising Parabacteroides goldsteinii.

Embodiment 2. The composition of embodiment 1, wherein bacteria have been removed from the composition.

Embodiment 3. The composition of any one of embodiments 1-2, wherein bacteria have been removed by filtration.

Embodiment 4. The composition of any one of embodiments 1-3, wherein the extracellular vesicles comprise proteins, nucleic acids, lipids, metabolites, and/or outer membrane of Parabacteroides goldsteinii.

Embodiment 5. The composition of any one of embodiments 1-4, wherein the extracellular vesicles are prepared using gel filtration, optionally followed by gradient ultracentrifugation.

Embodiment 6. A method of promoting immune tolerance comprising administering the composition of any one of embodiments 1-5 to a cell or comprising administering live Parabacteroides goldsteinii to a cell, wherein the immune tolerance of the cell is characterized by any one or more of increased IL-10 and/or adenosine production; activation of adenosine receptor A2a; increased expression of Il10, lipocalin-2 (Lcn2), and/or indoleamine 2, 3-dioxygenases (Ido1/2); reduced expression of (d40, Il12b, Il23a, Il1f9, and/or Il-36 gamma; increased autophagy; and reduced TNF-α, IL-4, and/or IL-5 production.

Embodiment 7. The method of embodiment 6, wherein the increased IL-10 production is stimulated all or partly through the pannexin1-CD73-A2a axis.

Embodiment 8. The method of embodiment 6 or embodiment 7, wherein the cell is a macrophage, T cell, or dendritic cell.

Embodiment 9. The method of embodiment 8, wherein the cell is a dendritic cell.

Embodiment 10. The method of embodiment 6, wherein the immune tolerance is characterized by increased adenosine production and/or activation of adenosine receptor A2a of the dendritic cell.

Embodiment 11. The method of any one of embodiments 6-10, wherein the cell is comprised in a cell culture and the administering is in vitro.

Embodiment 12. The method of any one of embodiments 6-10, wherein the cell is comprised in a subject and the administering is oral delivery of the composition.

Embodiment 13. The method of embodiment 12, wherein the administering does not change the composition of the subject's microbiome.

Embodiment 14. A method of preventing or treating an autoimmune or inflammatory disease comprising administering live Parabacteroides goldsteinii and/or the composition of any one of embodiments 1-5 or the product of embodiment 26 to a subject, optionally wherein the administering produces increased IL-10 and/or adenosine production; activation of adenosine receptor A2a; increased autophagy; and/or reduced TNF-α, IL-4, and/or IL-5 production, in one or more cells of the subject.

Embodiment 15. The method of embodiment 14, wherein the administering comprises administering live Parabacteroides goldsteinii to a subject and the administering changes the composition of the subject's microbiome.

Embodiment 16. The method of embodiment 14, wherein the administering comprises administering the composition of any one of embodiments 1-5 and the administering does not change the composition of the subject's microbiome.

Embodiment 17. The method of any one of embodiments 14-16, wherein the administering is by oral administration.

Embodiment 18. A method of preventing or treating an autoimmune or inflammatory disease comprising administering an A2a adenosine receptor agonist to a subject, optionally wherein the administering produces increased IL-10 production; increased autophagy; and/or reduced TNF-α, IL-4, and/or IL-5 production, in one or more cells of the subject.

Embodiment 19. A method of preventing or treating an autoimmune or inflammatory disease comprising administering a composition comprising (1) live Parabacteroides goldsteinii and/or the composition of any one of embodiments 1-5 and (2) an A2a adenosine receptor agonist to a subject, optionally wherein the administering produces increased IL-10 and/or adenosine production; activation of adenosine receptor A2a; increased autophagy; and/or reduced TNF-α, IL-4, and/or IL-5 production, in one or more cells of the subject.

Embodiment 20. The method of embodiment 18 or embodiment 19, wherein the A2a adenosine receptor agonist is adenosine, regadenoson, spongosine, sonedenoson (MRE-0094), apadenoson (ATL-146e, BMS 068645), evodenoson, ATL-313, DE-112, UK-371104 27, UK-432097, or GW328267X.

Embodiment 21. The method of any one of embodiments 18-20, wherein the administering increases levels of adenosine and/or IL10 in the subject.

Embodiment 22. The method of any one of embodiments 14-21, wherein the increased levels of adenosine and/or IL 10 are measured in the gut or synovial tissue or fluid.

Embodiment 23. The method of any one of embodiments 14-22, wherein the administering decreases levels of TNF-α, IL-4, and/or IL-5 in the subject.

Embodiment 24. The method of embodiment 23, wherein the decreased levels of TNF-α, IL-4, and/or IL-5 are measured in the gut or synovial tissue or fluid.

Embodiment 25. The method of any one of embodiments 14-24, wherein the autoimmune or inflammatory disease is rheumatoid arthritis (RA).

Embodiment 26. A method of producing extracellular vesicles from Parabacteroides goldsteinii comprising growing Parabacteroides goldsteinii in culture; collecting a sample comprising Parabacteroides goldsteinii cells and cell culture supernatant; removing Parabacteroides goldsteinii cells from the sample; performing gel filtration and gradient ultracentrifugation on the sample, wherein the gel filtration and gradient ultracentrifugation are separate steps performed in either order.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1H show that Parabacteroides goldsteinii ASF519 induces multifaced tolerogenic phenotypes. A, Measurement of cytokine production by BMDCs and Foxp3+ Treg proliferation in the coculture of BMDCs and splenocytes, using filter sterilized spent media (SSM) collected during bacterial growth as the stimuli of BMDCs. Mean values of three biological replicates are shown. B, Prominent immunophenotypes evoked by each bacterial member irrespective of bacterial growth stages. All blue cells present statistical significance (p<0.05, two-tailed Student t test) with at least a 35% change in cytokine levels or a 2-fold change in Treg proliferation. C, IL-10 production by cultured or enriched cell types after stimulation with either broth or ASF519 SSM (n=3-4). D, Experimental design for RNA sequencing of BMDCs that were exposed to ASF519 SSM and zymosan in a step-wise manner (n=3). E, Intersection of differentially expressed genes (DEGs) (adjusted p<0.01 and |fold change|>3) in two between-group comparisons, ASF519 versus Broth, and zymosan-involved ASF519_Zym versus Broth_Zym. F, Heatmap of select DEGs in either comparisons shown in E. The fold change of each gene was relative to the Broth condition. The bar plot indicates fold changes in ASF519_Zym versus ASF519, indicative of synergy (Syn.) and antagonism (Ant.) between ASF519 and zymosan. G and H, Synergy on IL-10 expression at the transcriptional (G) and cytokine level (H) in BMDCs exposed to monotonous (bacterial medium) or step-wise stimulation (bacterial medium+zymosan) (n=3). Data (c and g) are represented as mean+sd across biological replicates and analyzed by two-tailed Student t test (C) or two-way ANOVA (G and H) (*p<0.05; **p<0.01; ***p<0.001).

FIGS. 2A-2H show P. goldsteinii ASF519 induces IL-10 through activation of adenosine receptor A2a. A, qPCR for Il10 in small intestine (SI) and colon collected from germ-free (GF), ASF519 mono-colonized (ASF519m), and specific pathogen-free (SPF) mice (n=5-8). B, Signal density of identified cecal metabolites in GF versus ASF519m mice (n=5-8). MTA, methylthioadenosine, SAH, S-adenosylhomocysteine. C, Quantification of metabolites in ASF519 SSM and unused chemically defined medium by LC-MS/MS (two biological replicates). AMP, adenosine monophosphate. D, Effect of inhibition of adenosine forming enzyme AHCY and adenosine receptors (A1, A2a, A2b, A3) by specific antagonists on IL-10 induction of BMDCs in response to ASF519 SSM (n=3). E, Validation of A2a engagement in IL-10 induction. Wildtype (WT) and A2a−/− BMDCs were stimulated by fractionated ASF519 proteins or PBS (n=3). F, Experimental design to test the role of adenosine receptor A2a in ASF519-mediated immunomodulation in antibiotic-treated (ABX) mice (n=6-8). Mice that received two doses of live or killed ASF519 were intraperitoneally injected daily with the A2a antagonist, ZM 241385. G-H, Flow cytometry analysis of IL-10 producing CD103-CD11c+ (G) and IL-10 producing CD103+ CD11c+ (H) cells in colonic lamina propria of mice supplemented with live (ASF519) versus killed (Ctrl) bacteria. Data are represented as mean+sem and analyzed by multiple Mann-Whitney test (A-C) and two-way ANOVA along with Tukey's test (E, G and H) (*p<0.05;**p<0.01; ***p<0.001; ****p<0.0001).

FIGS. 3A-3M show that ASF519-derived extracellular vehicles (EVs) recapitulate immune tolerance via A2a. A, Bioanalytical strategies to identify responsible components in ASF519 SSM. B, Dose-dependent induction of IL-10 by enriched components after acetone precipitation (n=3). C, IL-10 inducing function of each fraction eluted from gel filtration of enriched ASF519 SSM. The UV absorbance of proteins (mAU) and IL-10 readouts were plotted against the elution volume and elution fractions, respectively. Data are representative of reproducible runs. D, IL-10 induction by fractions from gradient ultracentrifugation loaded with bioactive fractions of gel filtration. Data are representative of reproducible runs (technical replicates n=3). E, EVs identified in the most potent IL-10 inducing fraction by electron microscopy (EM). F, EV formation (arrow) during ASF519 growth revealed by EM. G, Production of IL-10 and TNF-α by BMDCs treated with an increasing dose of purified EVs (n=4). H, Validation of A2a engagement in IL-10 induction. Wildtype (WT) and A2a−/− BMDCs were stimulated by ASF519 EVs or PBS (n=3). I, Flow cytometry analysis of IL-10 producing CD103+ CD11c+ cells in colonic lamina propria of mice received i.p. injection of purified ASF519 EVs versus PBS. Measurement of IL-10 (J) and TNF-α (K) in the supernatants of zymosan-stimulated lamina propria cells that were extracted from EV versus PBS treated mice. Data are represented as mean+sd and analyzed by Student t tests (B, C, and H) and Welch's test (I-K) (*p<0.05, **p<0.01; ***p<0.001). L, abolishment of IL-10 induction by treatment of ASF519 SSMs with immobilized protease K digestion. A defined medium was used to grow ASF519 to generate SSMs. Vehicle or heat-killed protease K treatment did not affect IL-10 induction. n=3, student t test, **P<0.01. M, a reduction in Treg proliferation when splenocytes were cocultured with BMDCs after stimulated with protease K treated versus non-treated SSMs. n=3, student t test, *P<0.05, *** P<0.001.

FIGS. 4A-4G show involvement of autophagy in EV-mediated IL-10 induction. A, Time-course measurement of extracellular ATP in in vitro assays with an increasing dose of purified ASF519 EVs (n=4). Assays without BMDCs or EVs are controls. B and C, Inhibition of adenosine producing ecto-nucleosidase CD73 by PSB-12379 (B) and ATP-releasing pannexin-1 by 10PanX using a scrambled peptide as control (C) in EV-treated BMDCs. D and E, Blockage of autophagy at the stage of autophagosome formation by 3-methyladenine (D) and autophagosome-lysosome fusion by hydroxychloroquine (E) in EV-treated BMDCs. F, Effect of Atg16L1 knockout in CD11c+ DC cells on IL-10 production. G, Model of ASF519 OMV-mediated regulation of IL-10 induction in BMDCs partly though autophagy-dependent pannexin-1-CD73 axis. The dashed arrows represent multiple steps involved. Ado, adenosine. Data are represented as mean+sd (n=3-4 in A-F) and analyzed by two-way ANOVA (F) (*p<0.05, **p<0.01;***p<0.001).

FIGS. 5A-5L show that P. goldsteinii ASF519 supplementation ameliorates disease with severity in mouse models of type 1 diabetes (T1D) and collagen-induced arthritis (CIA) and that oral administration of purified ASF519 EVs prevents collagen-induced arthritis dependent on A2a signaling. A, Experimental design to test the effect of orally administered heat-killed (Ctrl) or live ASF519 on T1D in female NOD mice (n=7-8). B, The percentage of mice grouped by non-fast blood glucose levels at 14-wk old. C, Histological insulitis scores of pancreatic islets in Ctrl and ASF519 mice. Each group has at least 120 islets assessed. The insulin producing function of insulitis free islets was validated by immunohistochemical staining of insulin. D, Experimental design to test the role of ASF519 in the collagen-induced arthritis mouse model (n=5-6). 10-wk old DBA/1J mice received four doses of P. goldsteinii ASF519 or UV-killed ASF519 and then were immunized with collagen to induce arthritis. E, Comparison of ASF519 abundance in stools before gavage and 21 days post gavage by qPCR. F, Loss of body weight after immunization with collagen (n=5-6). G, Arthritis severity developed on four paws of immunized Ctrl and ASF519 mice indicating development of arthritis based on arthritis score with images of representative ASF519 or dead ASF519 recipient mice. H and I, Images of representative animal treated with ASF19 (H) or killed ASF19 (I) with notable inflammation on paws indicated by circles. Data for panels A-G are represented by mean+sem and the significance was tested by Mann-Whitney U test (E) and two-way ANOVA (F-G) (**p<0.01, ***p<0.001). J, Experimental design to test the effect of orally administered ASF519 EVs versus PBS and A2a receptor blockage by ZM241385 on collagen induced arthritis in DBA mice. Oral and intraperitoneal (i.p.) treatment was done tri-weekly at the same time. K, Arthritis severity developed on four paws of each mouse after collagen immunization. L, Change in body weight (%) of mice before and after immunization. Data are represented by mean+sem and the significance was tested by two-way ANOVA (B and C) (n=6-9, *p<0.05, ***p<0.001, ****p<0.0001).

FIGS. 6A-6D show the in vitro immunoregulatory profiles of ASF members. A, phylogeny of ASF members in the context of human gut microbiome. B, Experimental design to test the effect of each ASF member on representative immunophenotypes of interest. Spent media were collected longitudinally during the growth of each member, then filter sterilized for stimulation of BMDCs followed by additional exposure to zymosan (named step-wise stimulation). Cytokine levels in the supernatant of treated BMDCs were measured by ELISA kits. Treated BMDCs were co-cultured with isolated splenocytes to evaluate their impact on Treg proliferation by flow cytometry. C, profiles of bacterial growth and production of IL-10, TNF-α, and TGF-β by BMDCs post step-wise stimulation with spent media collected at the indicated time. All strains were cultured in BHI and ASF519 was also cultured in the defined medium (DM). All cytokine measurements have at least 3 biological replicates. D, Flow cytometry analysis of Treg proliferation in the coculture of cell tracer labeled splenocytes and stimulated BMDCs. Uninoculated broth control and ASF519 SSM of 23 hours bacterial growth were used as stimuli (n=3).

FIG. 7 shows the prevalence of Parabacteroides goldsteinii in previous human microbiome project (HMP) studies. The inset violin plot demonstrates statistics of the prevalence data across HMP studies.

FIGS. 8A-8C show characteristics of Parabacteroides goldsteinii ASF519 mediated immunoregulation. A, Effect of TNF signaling blockage on the production of IL-10 and TNF-a by step-wisely stimulated BMDCs. B and C, Effect of stimulation time on the production of IL-10 (B) and TNF-a (C) by BMDCs that were monotonously stimulated by either BHI broth or ASF519 SSM. Data are represented as mean+sd (n=3) and analyzed by two-way ANOVA. The significance indicates the difference between curves.

FIGS. 9A-9I show RNA sequencing data analysis of BMDCs. A, Principle component analysis (PCA) of normalized gene counts in samples. Each condition has 3 biological replicates. B, Venn diagrams showing unique and shared DEGs (p<0.01 and |fold change|>3) in different comparisons. C and D, Volcano plots for DEGs found in different conditions. DEGs are shown. E, GO enrichment analysis of upregulated and downregulated DEGs in ASF519 SSM stimulated BMDCs without further exposure to zymosan. All listed GOs are significantly enriched (adj p<0.05). F, heatmap plot showing the relative expression level of immune function related genes by referring to Broth. Genes encode cytokines (left), cytokine receptors (middle), complement/co-stimulatory molecules and others (right). G-I, plots showing a synergistic or antagonistic effect of ASF519 SSM and zymosan on the transcript abundance (n=3 per condition, mean+sd, Mann-Whitney U test, **p<0.01, ***p<0.001).

FIGS. 10A-10C show in vivo phenotypes in gnotobiotic mice. A, qPCR analysis to interrogate the effect of ASF519 monoassociation (ASF519) on the transcript abundance of genes associated with immune functions, epithelial integrity, and mucin synthesis in small intestine or colon. Germ-free (GF) tissues were used. Data was normalized to the gapdh gene in GF tissues. B and C, measurement of IL-4 (B) and IL-5 (C) in sera of GF, ASF519m, and SPF mice. Data are represented as mean+sem (n=4-8 in A-C) and analyzed by Mann-Whitney test (*p<0.05).

FIGS. 11A-11D show analysis of metabolome data. A, Volcano plot showing untargeted metabolomes in cecal contents of germ-free (GF) and ASF519 mono-colonized (ASF519m) mice. Significantly changed m/z features were labeled (adjusted P<0.001 and |fold change|>3). The signal intensity of 1068 m/z features was increased by ASF519 colonization, whereas 1744 m/z features decreased. B, List of metabolic pathways that were significantly influenced by ASF519 mono-association (gamma adjusted p<0.05). C, Effect of ASF519 mono-association (ASF519m) in gnotobiotic mice on metabolic pathways through functional analysis of cecal metabolome data. The metabolism of 13 amino acids (e.g., Cys and Met), galactose (Gal), and amino and nucleotide sugars were significantly influenced by ASF519m versus GF mice. D, adenosine derivatives quantified in ASF519 SSM and unused chemically defined medium (two biological replicates).

FIGS. 12A-12J show involvement of adenosine receptor A2a in IL-10 immunophenotype. A, Blockage of adenosine receptor A2a disabled IL-10 induction of BMDCs that were stimulated by BHI broth, crude ASF519 SSM, or 0.75 μg/ml bioactive fractions of gel filtration. The AHCY inhibitor 3-deazeadenosine (DZA) to block the formation of adenosine, A2a antagonist ZM 241385 (ZM), and A2b antagonist MRS-1754 (MRS), were used here. Mean+sd, n=3, Mann-Whitney U test, *p<0.05; **p<0.01; ***p<0.001. B, Measurement of intracellular cyclic AMP in BMDCs post step-wise stimulation. BMDCs were firstly stimulated by PBS control or bioactive fraction (0.75 μg/ml) before adding zymosan. Data are represented as mean+sd (n=2) and analyzed by two-tailed Student t test (*p<0.01). C-G, Flow cytometry analysis of colonic lamina propria cells extracted from mice supplemented with live versus killed (Ctrl) ASF519 and meanwhile treated with the A2a antagonist ZM 241385. H, Flow cytometry analysis of colonic lamina propria cells extracted from mice treated by i.p. injection of purified EV versus PBS. Data are represented as mean+sem (n=6-8 in C-G) and analyzed by two-way ANOVA along with Tukey's test (C-G) and Welch's test (H-J) (*p<0.05;**p<0.01). I and J, qPCR for adenosine receptors in small intestine (I) and colon (J) of mice treated by i.p. injection of purified EV versus PBS.

FIGS. 13A-13E show identification of microbial components responsible for IL-10 induction. A, Effect of heat treatment of ASF519 SSM and uninoculated broth on IL-10 induction (n=3, mean+sd, Mann-Whitney U test, ***p<0.001). B, Effect of immobilized protease K (ProK) treatment of ASF519 SSM on IL-10 induction in BMDC assays (n=3). The heat killed ProK and ProK vehicle were used as controls (n=3, mean+sd, Mann-Whitney U test, ***p<0.001). C, Experimental workflow to identify responsible molecules. Two protein enrichment approaches (ammonium sulfate and cold acetone) have been tested to concentrate ASF519 SSM from rich BHI medium or defined medium. Two types of gel filtration columns have been tested before ultracentrifugation and analyses. D, IL-10 inducing function of fractions eluted from HiPrep 16/60 Sephacryl S400 HR loaded with ASF519 SSM developed from the defined medium. The UV absorbance of proteins (mAU) and IL-10 readouts were plotted against the elution volume (Ev.) and elution fractions. The gel filtration standard was run under the same condition to estimate the molecular size. The bioactive fractions were eluted in the excluded volume. E, IL-10 production by BMDCs firstly treated with purified OMVs, immobilized proteinase K-treated OMVs, or Tris-HCl, then exposed or not exposed to zymosan for additional stimulation (n=4, mean+sd, t test, *p<0.05, ***p<0.001).

FIGS. 14A-D show effects of EV treatment on BMDCs. A, The extracellular ATP signal ratio of EV-treated BMDCs to non-treated BMDCs at each time point. B, Impact of EV treatment on the membrane permeability of BMDCs through measuring the lactate dehydrogenase activity (LDH) in the extracellular space. Lysate of BMDCs was used as the positive control. C, Impact of EV treatment on apoptosis, which was quantified by flow cytometry analysis. D, RNA-seq data of mapilc3a in BMDCs treated step-wisely, first with unused broth or ASF519 SSM, then with zymosan. Data are represented as mean+sd (n=3 in B and C) and analyzed by two-way ANOVA (C) (*p<0.05, ***p<0.001).

FIG. 15 shows gating strategy for flow cytometry analysis.

FIG. 16 shows that spent media of Parabacteroides goldsteinii induce the production of IL-10 but not TNF-α. The IL-10 level released by stimulated bone marrow derived dendritic cells (BMDCs) with filter-sterilized spent media (SSMs) at different bacterial growth stages, corelates with ASF519 growth as indicated by cell biomass; in contrast, TNF-a displays a reverse correlation. The IL-10/TNF-α ratio was increased during bacterial growth. SSMs of ASF519 cultured either in rich brain heart infusion broth (BHI) or defined medium (DM) yielded similar trends. BMDC assays at each time point have 3-4 replicates.

FIGS. 17A-17D show that spent media of Parabacteroides goldsteinii promote CD4⁺FoxP3⁺ Treg expansion. Compared with using BHI broth as stimulus (A and B), BMDCs exposed to ASF519 SSMs (C and D) increase the proportion of CD4+FoxP3+ T cells in the cocultured splenocytes and dramatically promote the expansion of CD4⁺FoxP3⁺ T cells.

### DESCRIPTION OF THE EMBODIMENTS

### I. Definitions

As used herein, “Parabacteroides goldsteinii” refers to a Gram-negative, obligate anaerobe that has isolated from human microbiome samples. As used herein, “Parabacteroides goldsteinii ASF519” or “Parabacteroides goldsteinii sp. ASF519” refers to Parabacteroides goldsteinii comprised in the ASF519 consortia. Parabacteroides goldsteinii ASF519 is reference NCBI:txid1235803 with GenBank ID No: AQFV00000000.2.

As used herein, “an autoimmune disease” refers to a disease initiated by a subject's immune system attacking the subject, such as an organ within the body. As used herein, “an inflammatory disease” refers to a disease characterized by chronic inflammation of one or more system or organ of a subject. Rheumatoid arthritis (RA) is a representative autoimmune or inflammatory disease.

### II. Compositions Prepared from Parabacteroides goldsteinii

In some embodiments, a composition is prepared from Parabacteroides goldsteinii. In some embodiments, the composition comprises live bacteria, spent media, culture supernatant extracts, and/or extracellular vesicles (EVs). The composition may be prepared from a sample of Parabacteroides goldsteinii that has been cultured in vitro.

In some embodiments, a composition comprises extracellular vesicles prepared from a sample comprising Parabacteroides goldsteinii. EVs refer to vesicles released from Parabacteroides goldsteinii that contain components from their mother bacteria. For bacteria comprised in a subject microbiome, EVs are thought to mediate communication between bacteria and its host via bioactive molecules including proteins, nucleic acids, lipids, and metabolites. EVs prepared from bacteria have been described, such as in US 2022/0296654, US 2020/0254028, and WO 2021/133904. The components comprised within an EV may be referred to as “EV cargo.” In some embodiments, the EV comprises proteins, nucleic acids, lipids, metabolites, and/or outer membrane of Parabacteroides goldsteinii. In some embodiments, EVs can mediate signaling by adhering or fusing with a host cell and allowing release of components within the EV to the cytoplasm of a host cell. Bacterial EVs can range from 20 to 400 nm in diameter.

In some embodiments, the EVs are isolated EVs that do not comprise bacteria. In some embodiments, EVs are prepared from a sample comprising Parabacteroides goldsteiniiusing gel filtration, ultracentrifugation, and/or sucrose or iodixanol gradient ultracentrifugation. In some embodiments, extracellular vesicles are prepared using gel filtration followed by gradient ultracentrifugation. Such a two-step purification method for EVs with gel filtration and gradient ultracentrifugation, in either order, can produce vesicles with higher purity than single-step purification methods.

In some embodiments, bacteria have been removed from a composition comprising EVs. In some embodiments, a sample comprising Parabacteroides goldsteinii is filtered (such as with a 0.22 μm filter) to eliminate bacteria before preparing EVs.

In some embodiments, a method of producing extracellular vesicles from Parabacteroides goldsteinii comprises growing Parabacteroides goldsteinii in culture; collecting a sample comprising Parabacteroides goldsteinii cells and cell culture supernatant; removing Parabacteroides goldsteinii cells from the sample; and performing gel filtration and gradient ultracentrifugation on the sample, wherein the gel filtration and gradient ultracentrifugation are separate steps performed in either order.

### III. Methods of Use of Compositions Comprising Extracellular Vesicles Prepared from Parabacteroides goldsteinii

In some embodiments, a composition as described herein may be administered to a cell. In some embodiments, the cell is a cell in culture. In some embodiments, the cell may be a cell comprised in a subject. For example, a cell may be an immune cell of a subject.

In some embodiments, EVs are administered to a cell, and EV cargo is delivered into the cell. In some embodiments, EVs are administered to a subject, and EV cargo is delivered into a subject (i.e., host) cell.

In some embodiments, administering a composition prepared from Parabacteroides goldsteinii or administering live Parabacteroides goldsteinii leads to immune tolerance of a cell. In some embodiments, the composition for administering comprises EVs prepared from Parabacteroides goldsteinii. In some embodiments, live Parabacteroides goldsteinii is administered. As used herein, “immune tolerance” refers to a reduced state of inflammation of a cell. In some embodiments, immune tolerance is characterized by increased IL-10 and/or adenosine production; activation of adenosine receptor A2a; increased expression of Il10, lipocalin-2 (Lcn2), and/or indoleamine 2, 3-dioxygenases (Ido1/2); reduced expression of ('d40, Il12b, Il23a, Il1f9, and/or Il-36 gamma; increased autophagy; and/or reduced TNF-α, IL-4, and/or IL-5 production.

In some embodiments, the method further comprises adhering or binding the extracellular vesicles to the cell after the administering and incorporating the contents of the extracellular vesicles into the cytoplasm of the cell.

In some embodiments, the increased IL-10 production is stimulated all or partly through the pannexin1-CD73-A2a axis.

In some embodiments, the cell is an immune cell. In some embodiments, the cell is a macrophage, T cell, or dendritic cell. In some embodiments, the cell is a dendritic cell. In some embodiments, the immune tolerance is characterized by increased adenosine production and/or activation of adenosine receptor A2a of the dendritic cell.

In some embodiments, the cell is comprised in a cell culture and the administering is in vitro. In some embodiments, the cell is comprised in a subject and the administering is oral delivery of the composition. In some embodiments, the administering does not change the composition of the subject's microbiome.

### IV. Methods of Prevention or Treatment of Autoimmune of Inflammatory Diseases by Administration of Live Parabacteroides goldsteinii or Extracellular Vesicles Prepared from Parabacteroides goldsteinii

In some embodiments, live Parabacteroides goldsteinii may be administered to a subject. In some embodiments, administering live Parabacteroides goldsteinii alters the immune response of the subject.

In some embodiments, extracellular vesicles prepared from Parabacteroides goldsteinii may be administered to a subject. In some embodiments, administering extracellular vesicles prepared from Parabacteroides goldsteinii alters the immune response of the subject.

In some embodiments, live Parabacteroides goldsteinii or extracellular vesicles prepared from Parabacteroides goldsteinii are orally administered to a subject to alter the immune response of the subject. Many commonly prescribed agents to alter the immune response must be administered by injection, and the presently described oral administration of live Parabacteroides goldsteinii or extracellular vesicles prepared from Parabacteroides goldsteinii has improved subject convenience.

In some embodiments, a method of preventing or treating an autoimmune or inflammatory disease comprises administering live Parabacteroides goldsteinii to a subject or extracellular vesicles prepared from Parabacteroides goldsteinii, wherein the administering produces increased IL-10 and/or adenosine production; activation of adenosine receptor A2a; increased autophagy; and/or reduced TNF-α, IL-4, and/or IL-5 production, in one or more cells of the subject. Representative experiments with administration of live Parabacteroides goldsteinii or extracellular vesicles prepared from Parabacteroides goldsteinii are presented in Example 8 below.

In some embodiments, the administering of live Parabacteroides goldsteiniichanges the composition of the subject's microbiome. In some embodiments, the administering of a composition comprising extracellular vesicles prepared from Parabacteroides goldsteiniidoes not change the composition of the subject's microbiome.

In some embodiments, the administering increases levels of adenosine and/or IL10 in the subject. In some embodiments, the increased levels of adenosine and/or IL 10 are measured in the gut or synovial tissue or fluid.

In some embodiments, the administering decreases levels of TNF-α, IL-4, and/or IL-5 in the subject. In some embodiments, the decreased levels of TNF-α, IL-4, and/or IL-5 are measured in the gut or synovial tissue or fluid.

### V. Methods of Prevention or Treatment of Autoimmune of Inflammatory Diseases by Administration of an A2a Adenosine Receptor Agonist

As described below in Example 3, the present studies indicated that adenosine was present in spent media from may be responsible for all or part of the immune tolerance seen after administration of the EVs.

In some embodiments, an A2a adenosine receptor agonist is administered to a cell to induce immune tolerance. As used herein, “an A2a adenosine receptor agonist” comprises any pharmacologic agent that partially or fully activates the A2a adenosine receptor. As such, an administration of A2a adenosine receptor agonist can mediate some physiological effects normally seen after administration of adenosine. In some embodiments, administration of an A2a adenosine receptor agonist mediates increased IL-10 production; increased autophagy; and/or reduced TNF-α, IL-4, and/or IL-5 production.

A variety of A2a adenosine receptor agonists are known in the art (see, for example, Trevethick et al., British Journal of Pharmacology 155:463-474 (2008); Muller et al., Biochim Biophys Acta 1808(5):1290-1308 (2011); and Valls et al., Biochem Pharmacol. 77(7):1117-1124 (2009)). In some embodiments, the A2a adenosine receptor agonist is adenosine, regadenoson, spongosine, sonedenoson (MRE-0094), apadenoson (ATL-146e, BMS 068645), evodenoson, ATL-313, DE-112, UK-371104 27, UK-432097, or GW328267X. In some embodiments, the administering increases levels of adenosine and/or IL10 in the gut or synovial fluid or tissue. In some embodiments, the administering decreases levels of TNF-α, IL-4, and/or IL-5 in the gut or synovial fluid or tissue.

### VI. Methods of Prevention or Treatment of Autoimmune of Inflammatory Diseases by Administration of an A2a Adenosine Receptor Agonist and Live Parabacteroides goldsteinii or Extracellular Vesicles Prepared from Parabacteroides goldsteinii

In some embodiments, a A2a adenosine receptor agonist is administered together with live Parabacteroides goldsteinii. In some embodiments, a A2a adenosine receptor agonist is administered together with extracellular vesicles prepared from Parabacteroides goldsteinii.

In some embodiments, a method comprises of preventing or treating an autoimmune or inflammatory disease comprising administering a composition comprising (1) live Parabacteroides goldsteinii or extracellular vesicles prepared from Parabacteroides goldsteinii and (2) an A2a adenosine receptor agonist to a subject, wherein the administering produces increased IL-10 and/or adenosine production; activation of adenosine receptor A2a; increased autophagy; and/or reduced TNF-α, IL-4, and/or IL-5 production in one or more cells of the subject.

In some embodiments, the administering increases levels of adenosine and/or IL10 in the gut or synovial fluid or tissue. In some embodiments, the administering decreases levels of TNF-α, IL-4, and/or IL-5 in the gut or synovial fluid or tissue.

### EXAMPLES

### Example 1. Division of Labor Among Murine Intestinal Microbes in In Vitro Immune Regulation

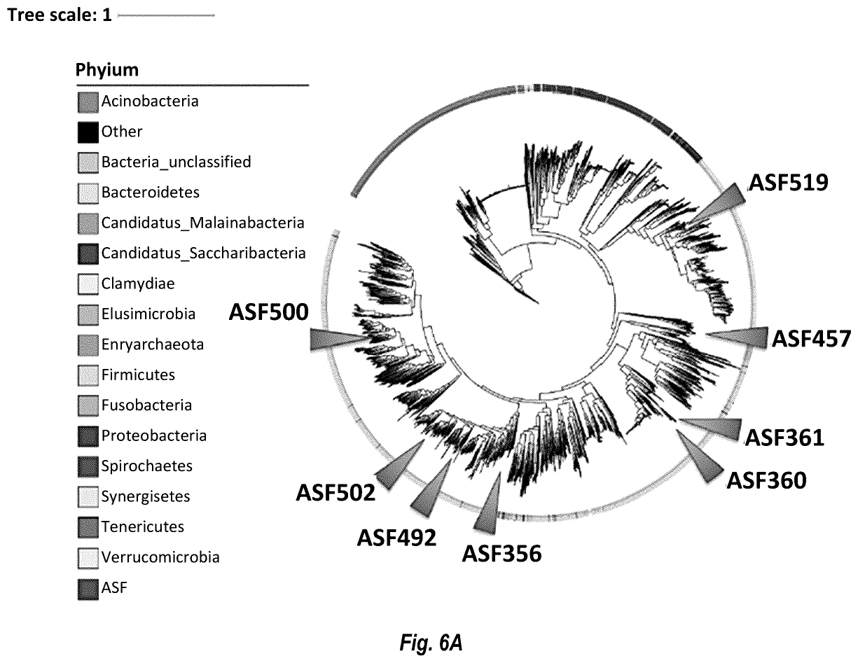

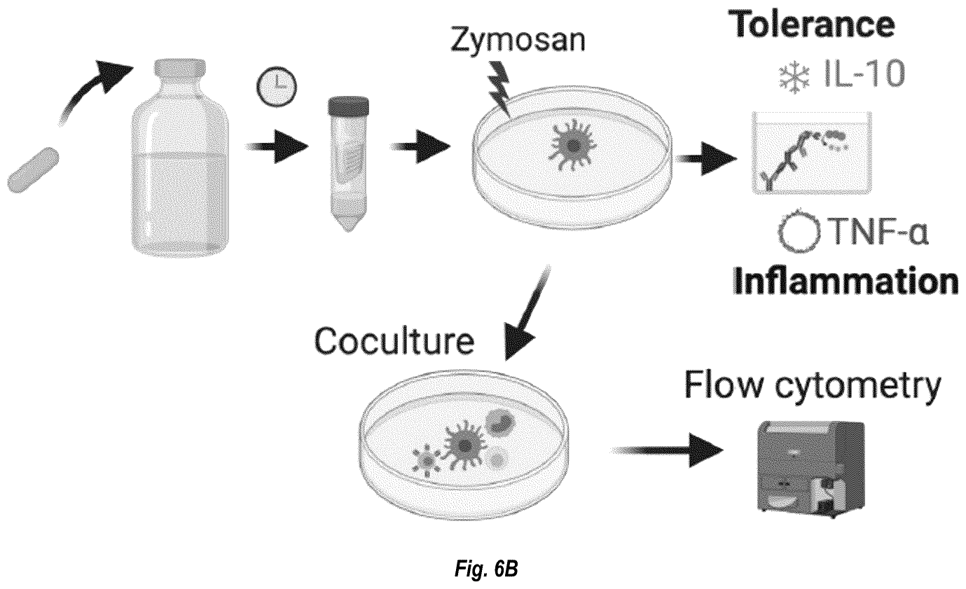

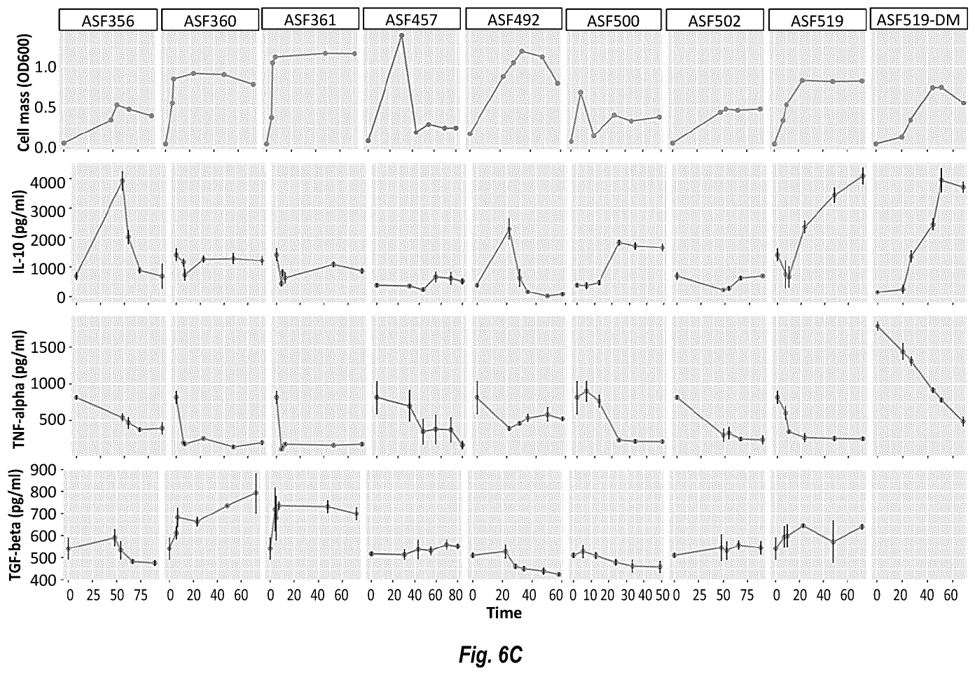

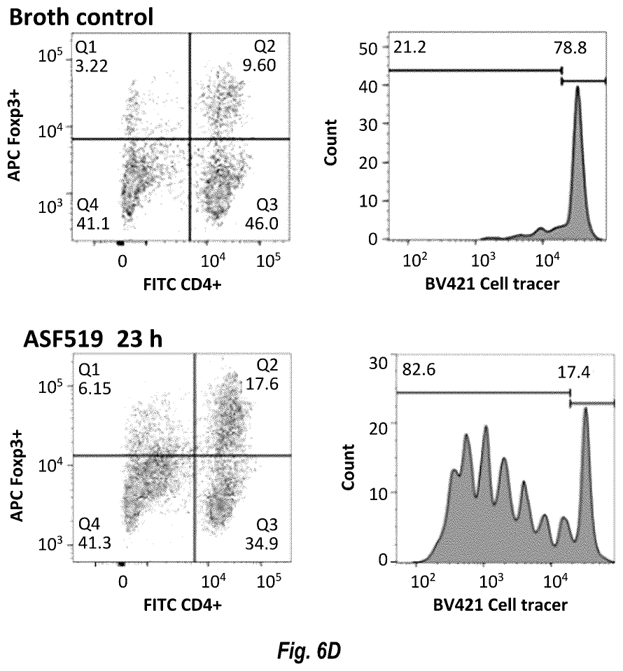

Synthetic microbial consortia consisting of characterized species have a lower complexity of inter-microbial and microbe-host interactions than a natural gut microbiota, but still approximately phenocopy immunological, metabolic, neurological, and other characteristics of animals with a natural gut microbiota (8). Examples include the 17-member Treg inducing Clostridia consortium (9), the 11-member IFNγ+ CD8+ T cell inducing bacterial assembly (10), and the multi-functional 8-member altered Schaedler flora (ASF) (5). The relative simplicity and cultivability of these “model microbiota” make them attractive tools to dissect the complex mechanistic functions of the human gut microbiome. The ASF is genetically competent in terms of gene richness (12) and its wide phylogenetic distribution (FIG. 6A), and importantly, it can resolve immunological, neurological, and metabolic defects in germ-free mice (13-21). It increases IL-10 production and promotes Treg proliferation in the colon (15) and is able to mimic the specific pathogen-free microbiota in reducing insulitis in MyD88−/−NOD mice (22). These results indicate a pronounced immune tolerance-eliciting function of this defined community, but the identity of the responsible microbes and molecules was not yet undetermined.

The role of each individual ASF member in a panel of immune regulation assays was studied to uncover a novel adenosine receptor A2a-dependent mechanism of immune tolerance mediated by microbial extracellular vesicles. These functional microbiome studies have the potential to open novel therapeutic avenues for the prevention and treatment of immune diseases.

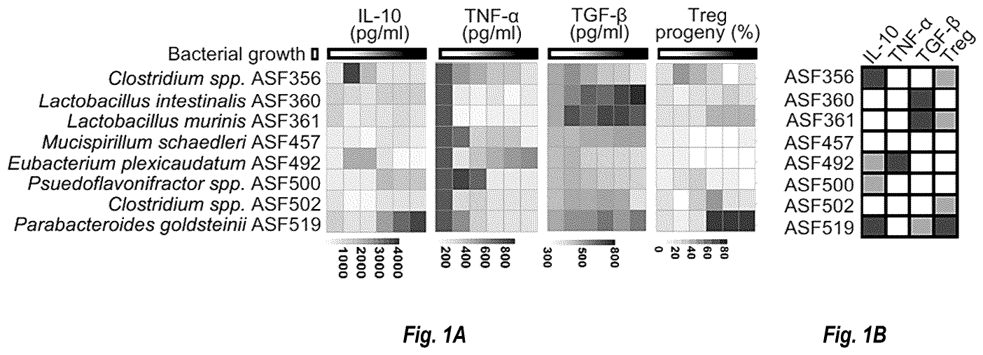

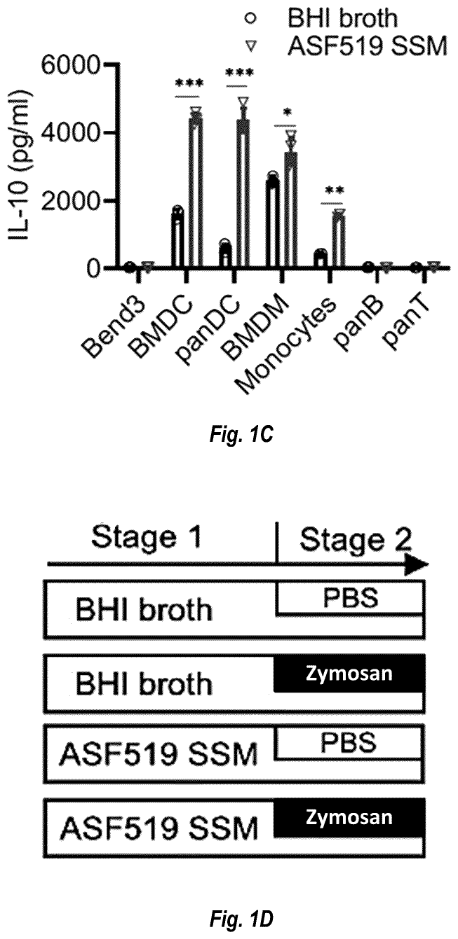

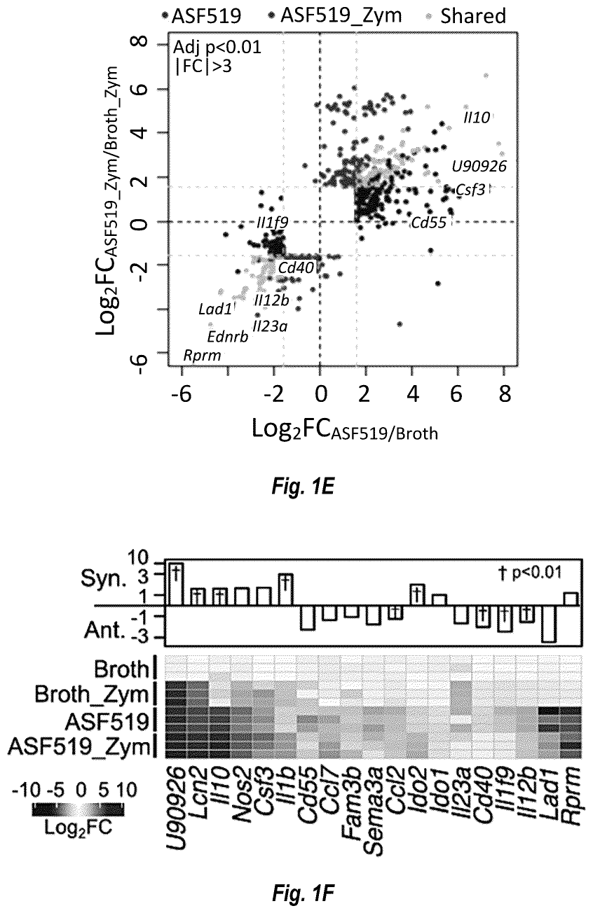

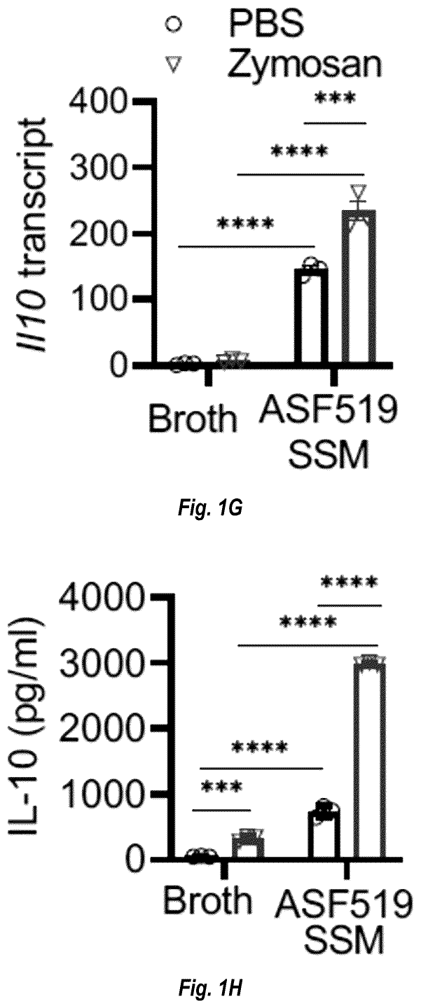

Dendritic cells (DCs) are professional antigen-presenting cells that play a critical role in inducing immunity and maintaining immune tolerance. Whether they are tolerogenic or immunogenic mainly depends on their cytokine production profiles and impact on T cells. The immunoregulatory potential of each ASF member was evaluated by stimulating bone marrow-derived dendritic cells (BMDCs) with filter-sterilized spent medium (SSM) collected during bacterial growth (FIG. 6B). SSM from Parabacteroides goldsteinii ASF519 dramatically induced the production of the anti-inflammatory cytokine IL-10 in a bacterial growth-dependent manner (FIG. 1A; FIG. 6C). In contrast, Clostridium spp. ASF356 only induced IL-10 at the exponential stage, while a weak IL-10 induction was observed with Eubacterium plexicaudatum ASF492 and Pseudoflavonifractor spp. ASF500. Measurement of another anti-inflammatory cytokine, TGF-β, revealed that Lactobacillus intestinalis ASF360 and Lactobacillus murinis ASF361 induce BMDCs to release this cytokine (FIG. 1A), which positively correlated with bacterial growth (FIG. 6C). By comparison, ASF519 had a mild effect on TGF-β expression. Although the uninoculated bacterial culture medium induces high levels of pro-inflammatory TNF-α, this effect was diminished by the mono-culture of 7 of the 8 ASF members (FIG. 1A). The exception was E. plexicaudatum ASF492, which displayed a TNF-α release when the stationary-phase SSM was used. These measurements revealed distinct cytokine-inducing profiles of ASF members coupled with growth phase-specific microbial physiology (FIG. 1A). Notably, ASF519 is the only member that induced two anti-inflammatory cytokines, IL-10 and TGF-β (FIG. 1B).

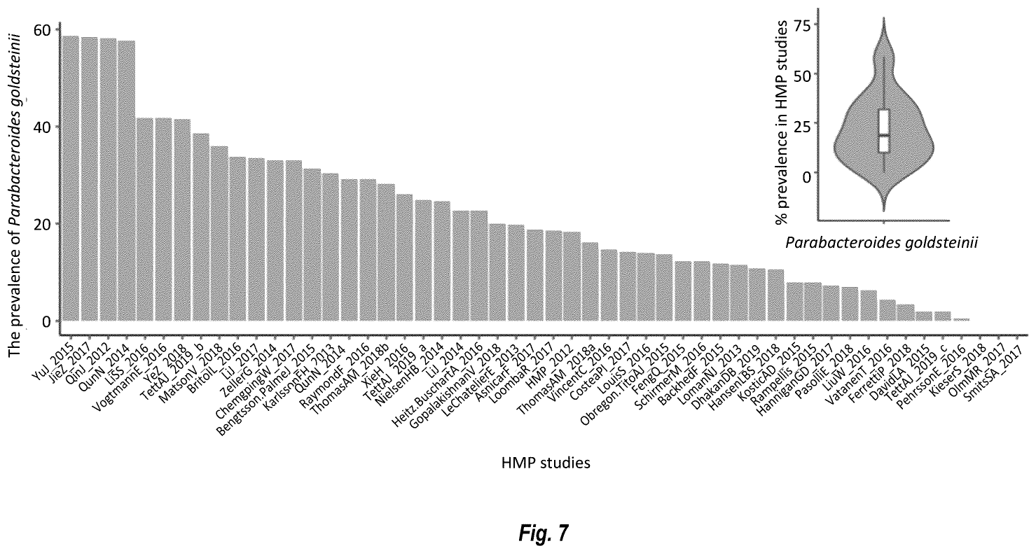

The impact of SSM-exposed BMDCs on Treg proliferation was evaluated via in vitro coculture with splenocytes from conventional mice (FIG. 6B). Interestingly, BMDCs exposed to ASF519 SSM, especially collected from late exponential and stationary phases, strongly promoted Foxp3+ Treg proliferation (80±6% by ASF519 SSM versus 20±9% by uninoculated bacterial culture medium) (FIG. 1A, FIG. 6D). The Foxp3+ Treg subset increased 1.8-fold when cocultured with the ASF519 SSM-stimulated BMDCs (FIG. 6D). A weak stimulatory effect on Treg proliferation was observed with three other members (ASF356, ASF361, and ASF502) but the effect peaked at distinct bacterial growth stages. Their impacts on both cytokine profiles and Treg proliferation demonstrate a “division of labor” among ASF members in regulating immunophenotypes of BMDCs (FIG. 1B). In particular, ASF519 promotes tolerogenic phenotypes of BMDCs by inducing anti-inflammatory cytokines and Treg proliferation. Considering the high abundance of ASF519 in the feces of ASF mice (5) and the relatively high prevalence (˜23%) of P. goldsteinii in the human gut microbiome according to The Human Microbiome Project (FIG. 7), its immunoregulatory activities were further studied.

### Example 2. Synergistic Regulation of Myeloid Cell-Specific IL-10 Induction

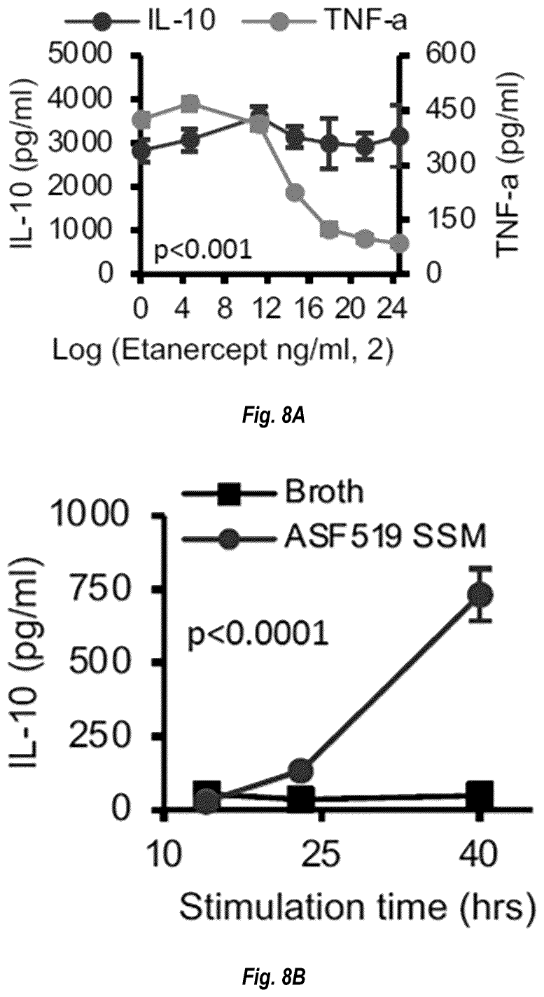

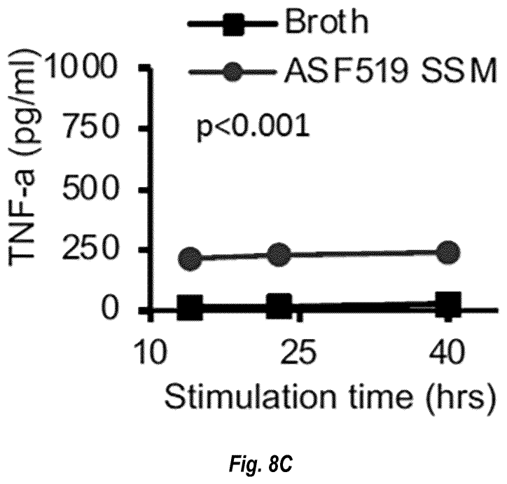

Because IL-10 is produced by a variety of immune cell types and is involved in both innate and adaptive immunity, the IL-10 inducing specificity of ASF519 was tested on a panel of diverse immune cells. Stimulation of isolated DCs, bone marrow-derived macrophages, and isolated splenic monocytes all yielded a high level of IL-10 (FIG. 1C); however, neither splenic panB nor panT stimulation, nor stimulation of immune-irrelevant BEND3 endothelial cells, showed a significant impact on IL-10 production. These data suggest that ASF519-mediated IL-10 induction is specific to myeloid-derived antigen-presenting cells. IL-10 induction was independent of TNF-α signaling (FIG. 8A), and it was a slow response, which ramps up after 24 hours of continuous stimulation of BMDCs by ASF519 SSM (FIG. 8B and 8C).

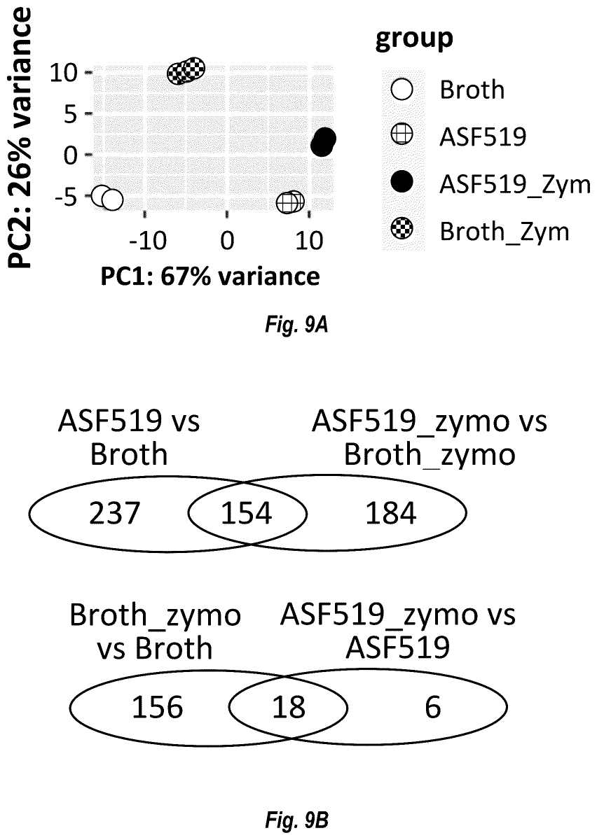

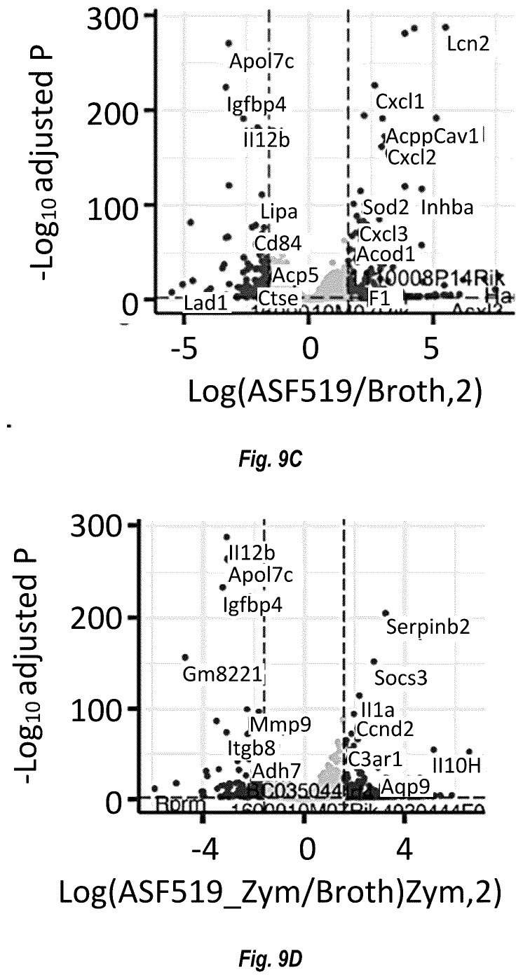

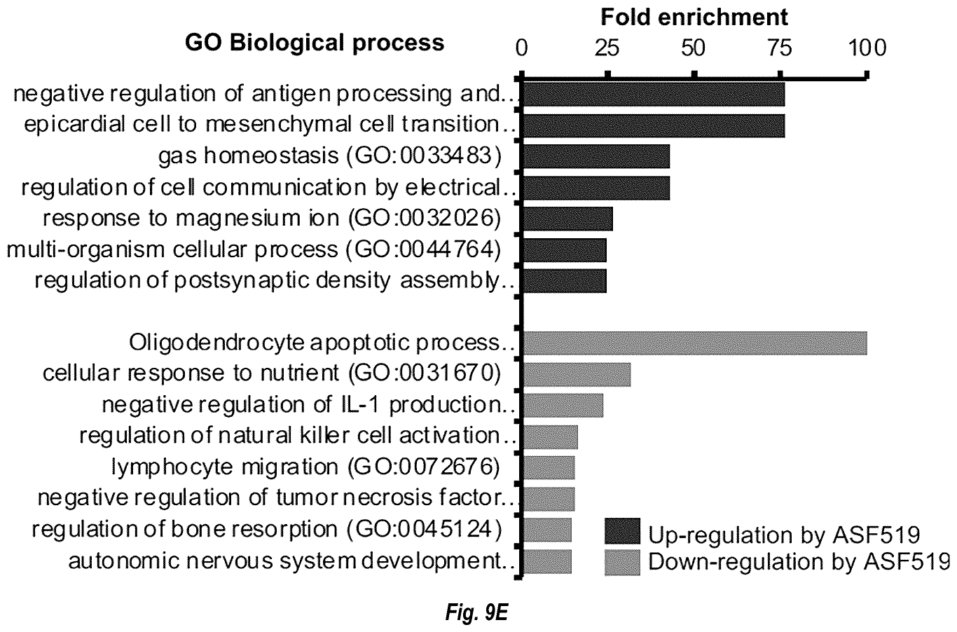

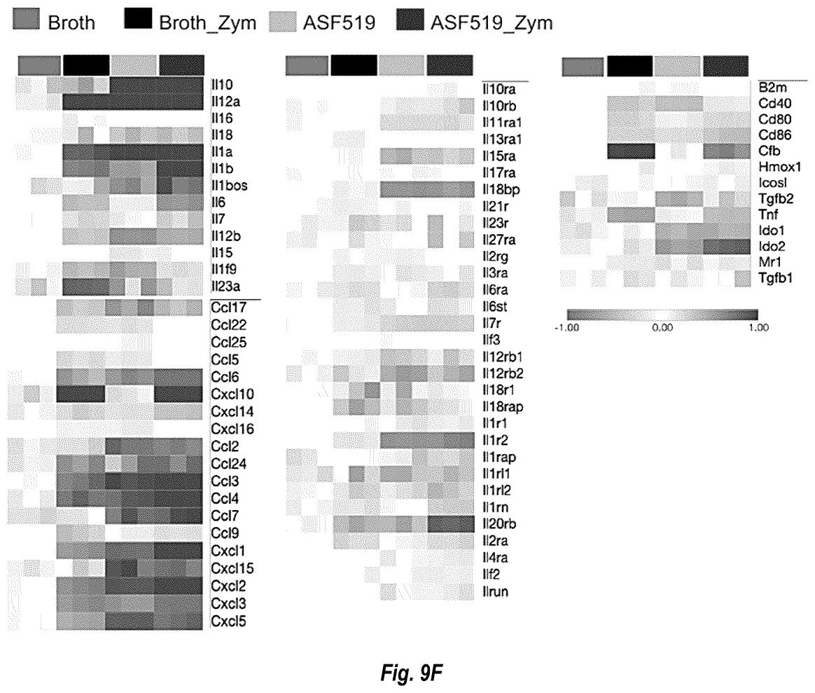

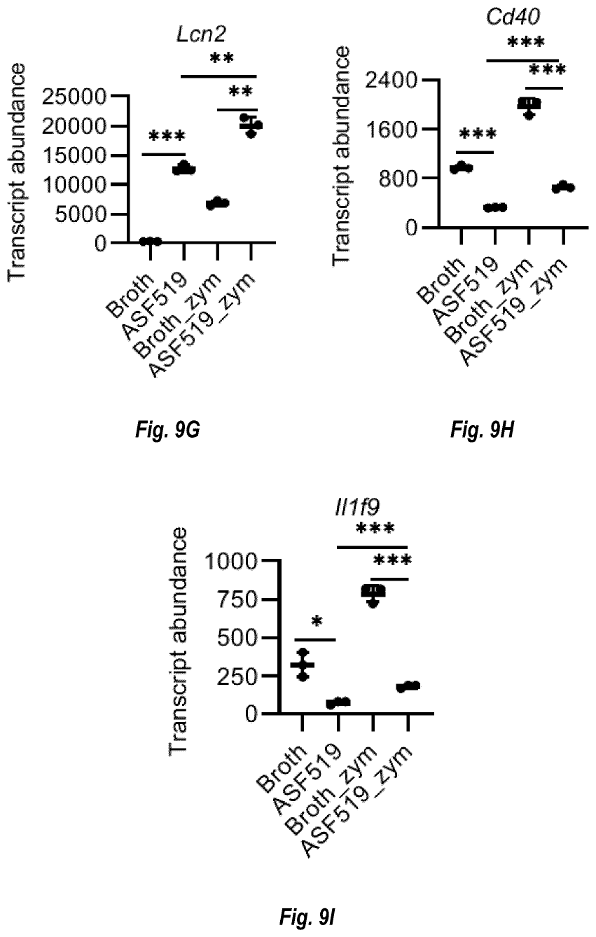

RNA sequencing of BMDCs revealed dramatic transcriptomic changes caused by stimulation with ASF519 SSM versus broth control (adjusted p<0.01, |fold change|>3) (FIG. 1D and 1E, FIG. 9A and 9B). Further stimulation by zymosan induced significant effects in broth-treated rather than ASF519 SSM-treated cells (FIG. 9B-9D), suggesting that ASF519 SSM may increase the tolerance of BMDCs to immunogenic zymosan. ASF519-induced differentially expressed genes have a wide association with the regulation of immune functions, cell communication, apoptosis, and other biological processes (FIG. 9E). Notably, negative regulation of antigen processing and presentation of peptide or polysaccharide antigen via MHC class II, and negative regulation of oligodendrocyte apoptotic process, are enriched most in upregulated and downregulated gene sets, respectively. At the gene level (FIG. 1F, FIG. 9F), a dramatic induction of tolerogenic Il10, lipocalin-2 (Lcn2), and indoleamine 2, 3-dioxygenases (Ido1/2) were seen, as well as a reduction of the co-stimulatory molecule Cd40 and inflammatory cytokines, including Il12b, Il23a, and Il1f9 (or Il-36 gamma). It is known that IL-36 gamma promotes inflammation and DC maturation; IL-12B and IL-23A together build the heterodimeric inflammatory IL-23 which is associated with autoimmune diseases and cancer. Their decreased transcript abundance may additively enhance the tolerogenic phenotypes of BMDCs. It is also notable that this step-wise stimulation with ASF519 SSM added before zymosan exerted a synergistic and antagonistic effect on certain transcripts (FIG. 1F; FIG. 9G-9I). For instance, Il10, Ido2, and U90926 were synergistically induced; whereas suppression of Il2b, Il1f9, and Cd40 by ASF519 SSM was attenuated by subsequent zymosan stimulation. This synergy on Il10 was seen at the protein level (FIG. 1G). Therefore, components of ASF519 SSM can induce multifaceted tolerogenic phenotypes of BMDCs in vitro.

### Example 3. ASF519 Colonization Induces Tolerogenic Phenotypes and Adenosine Accumulation

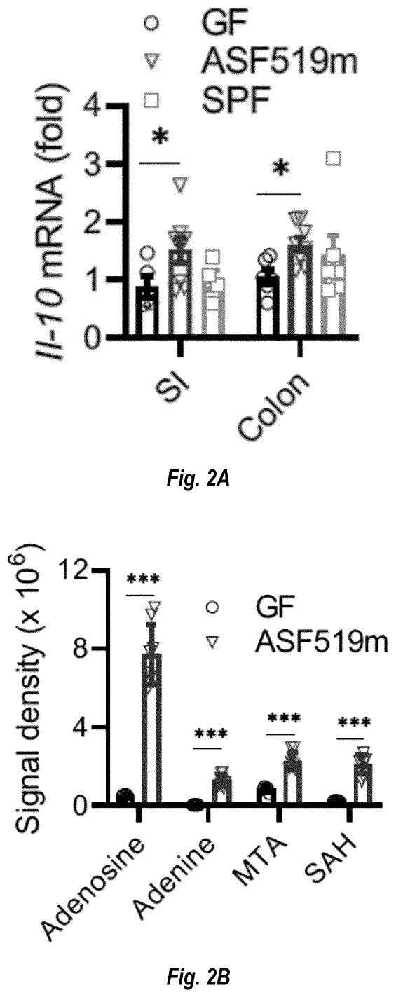

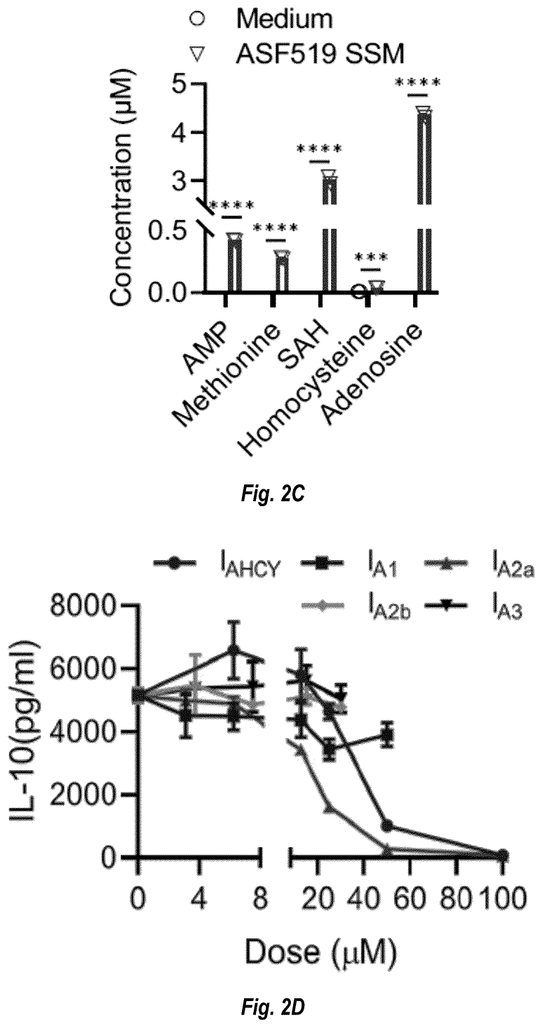

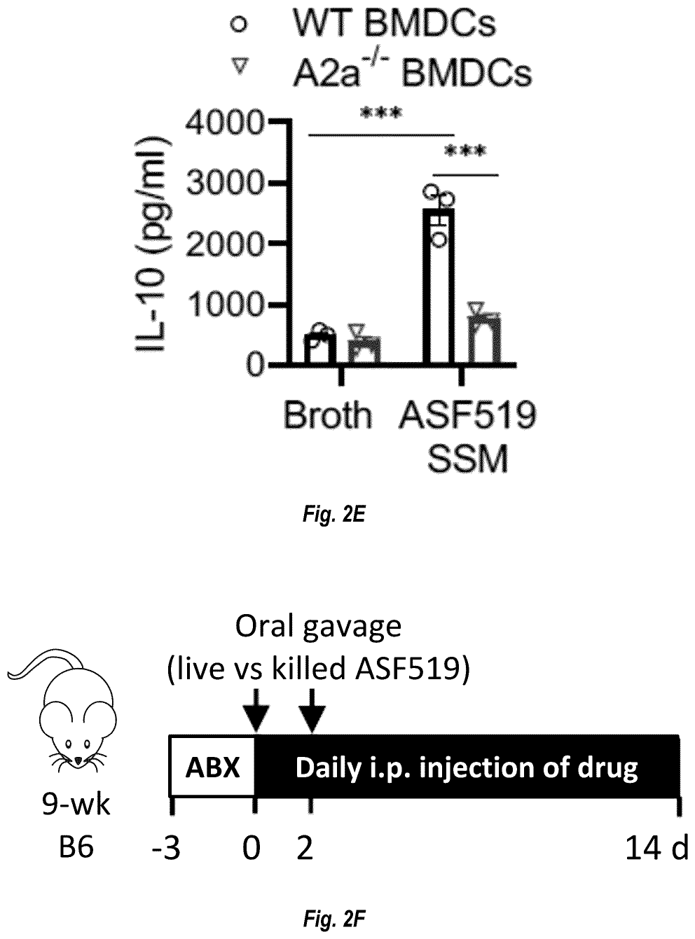

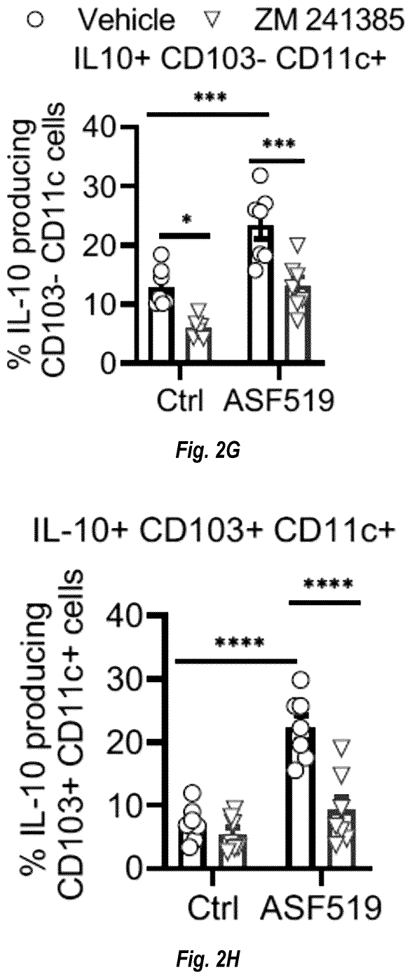

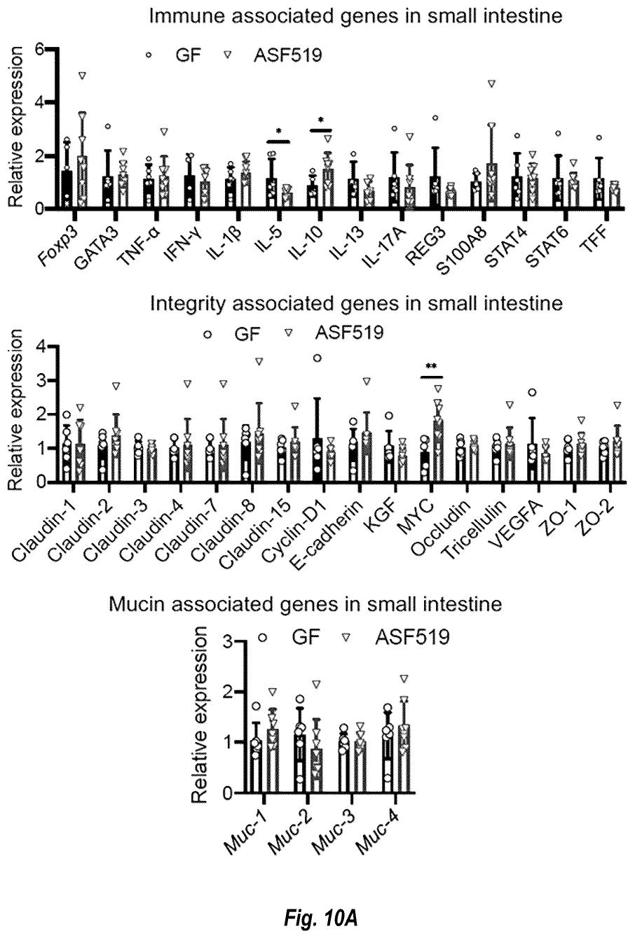

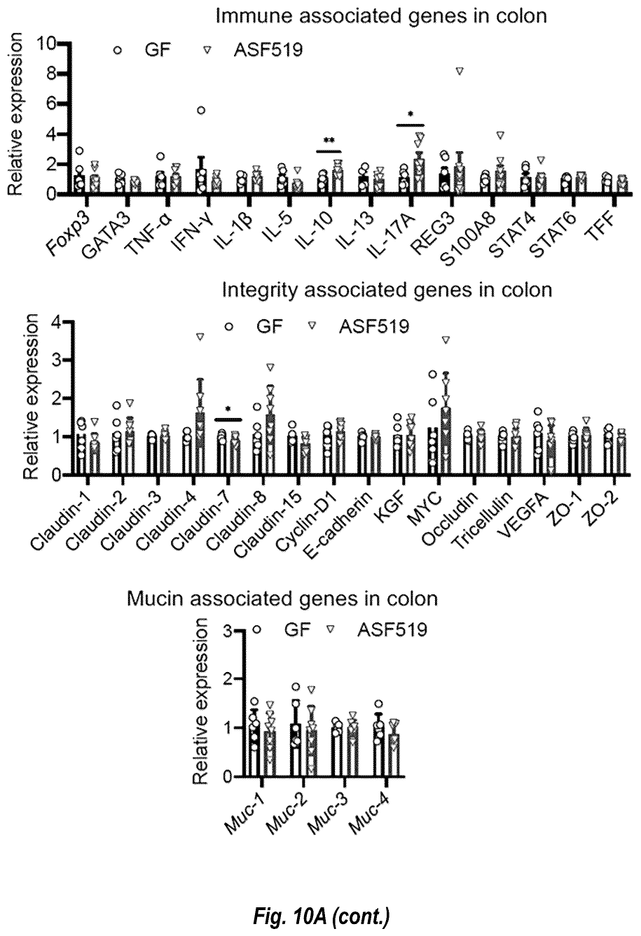

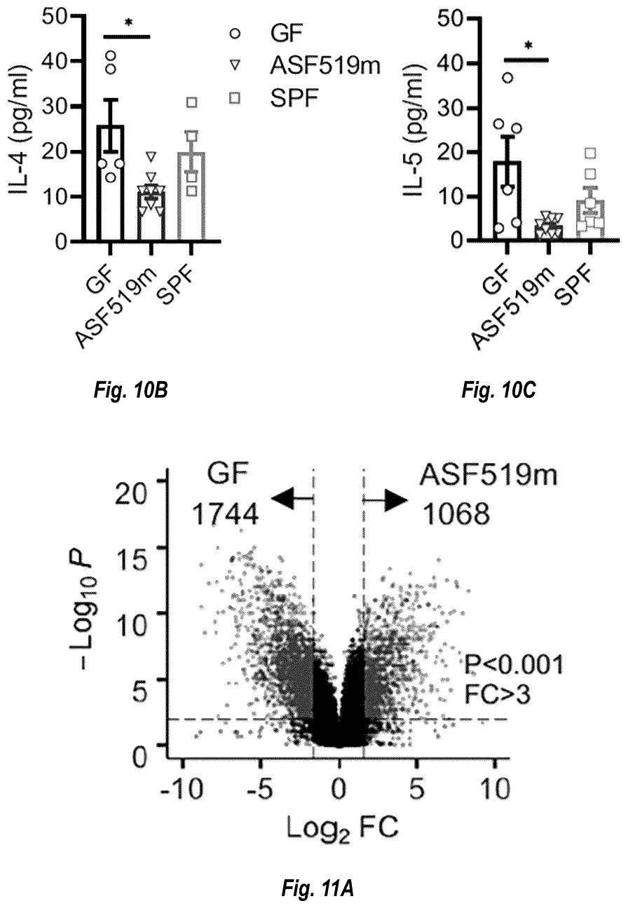

To probe in vivo immunoregulatory function, ASF519 was orally introduced to 4-week-old germ-free mice for a 2-week long mono-association. With qPCR analysis for 34 select genes involved in intestinal integrity, mucin synthesis, and immune functions, ASF519 mono-association was found to increase the transcript level of Il10 in the small intestine and colon (FIG. 2A) and Il17a in the colon, and it significantly reduced Il5 transcripts in the small intestine (FIG. 10A). Il10 up-regulation is consistent with ASF519 SSM-mediated IL-10 induction observed in vitro. Other genes involved in mucin synthesis and intestinal integrity were not influenced by ASF519, with the exception of an increase in the cell proliferation promoting myc gene in the small intestine (FIG. 10A). Measurement of 18 cytokines in sera revealed a systematic reduction of IL-4 and IL-5 in ASF519 mice (FIG. 10B and 10C). IL-5 is mainly secreted by activated T helper 2 cells which originally rely on IL-4 to differentiate from naive T cells, and both IL-4 and IL-5 are involved in the proliferation and function of activated B and T cells. Their concurrent reduction in circulation may imply the effects of ASF519 on systemic immune response.

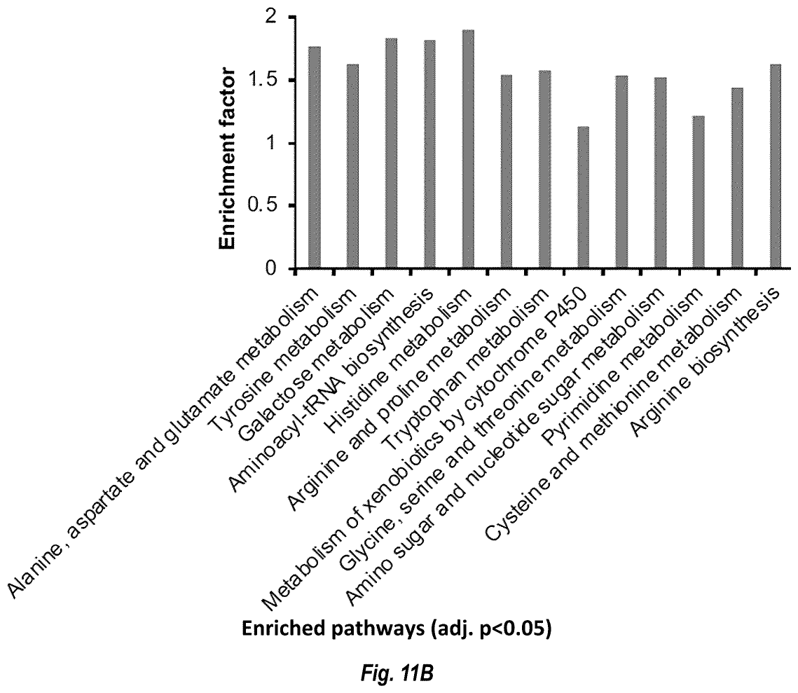

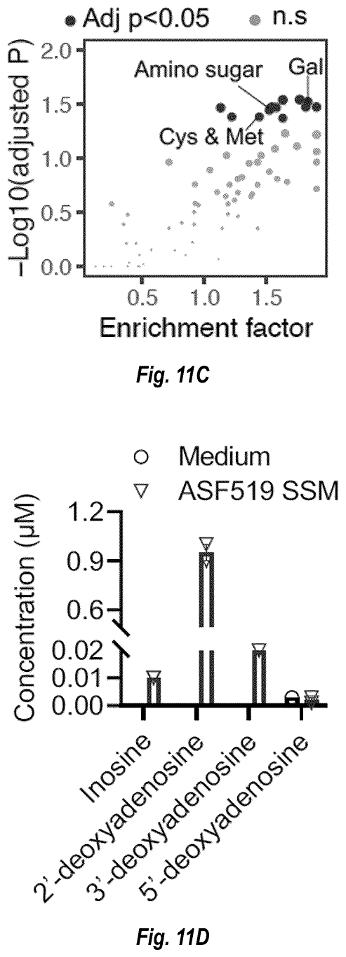

Untargeted metabolomics revealed vast changes in the abundance of cecal metabolites caused by ASF519 mono-association (FIG. 11A). These metabolites were significantly enriched for the metabolism of 13 amino acids, galactose, and amino and nucleotide sugars (FIG. 11B and 11C). Notably, ASF519 colonization increased cecal adenosine by 15-fold, as well as the adenosine-forming substrate S-Adenosyl-L-homocysteine (SAH) and one of the adenosine-degradation products, adenine, by 9-fold and 73-fold, respectively (FIG. 2B). The metabolome in ASF519 spent medium and unused chemically defined medium was evaluated. It is interesting that high levels of adenosine (4.2 μM), S-adenosylhomocysteine (SAH, 3.1 μM) (FIG. 2C), and a series of nucleoside derivatives (FIG. 11D), were detected in the spent medium, indicating the formation of adenosine during ASF519 growth. Therefore, ASF519 increases the presence of adenosine and related metabolites in vitro and in cecal contents of mice. As adenosine has a very short half-life in the body and its physiological level is in the nanomolar range in humans and mice (28), elevated adenosine formation could activate the adenosine signaling.

### Example 4. Microbial Activation of Adenosine Receptor A2a is Required for IL-10 Induction

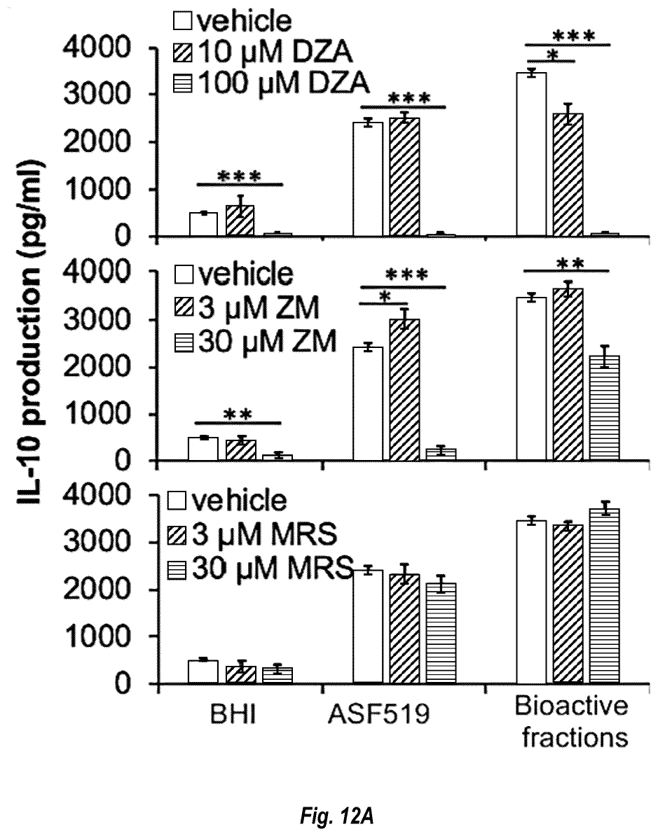

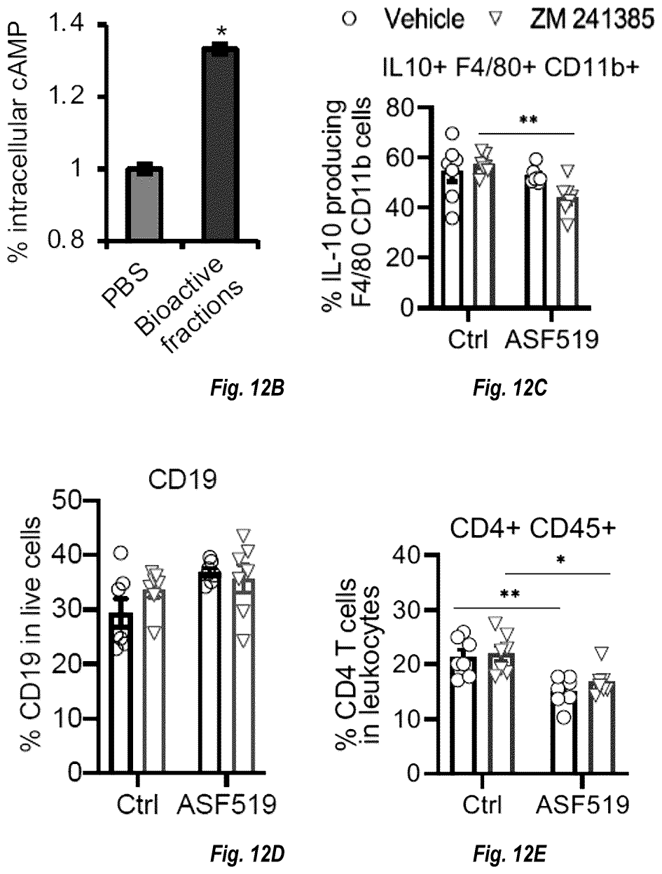

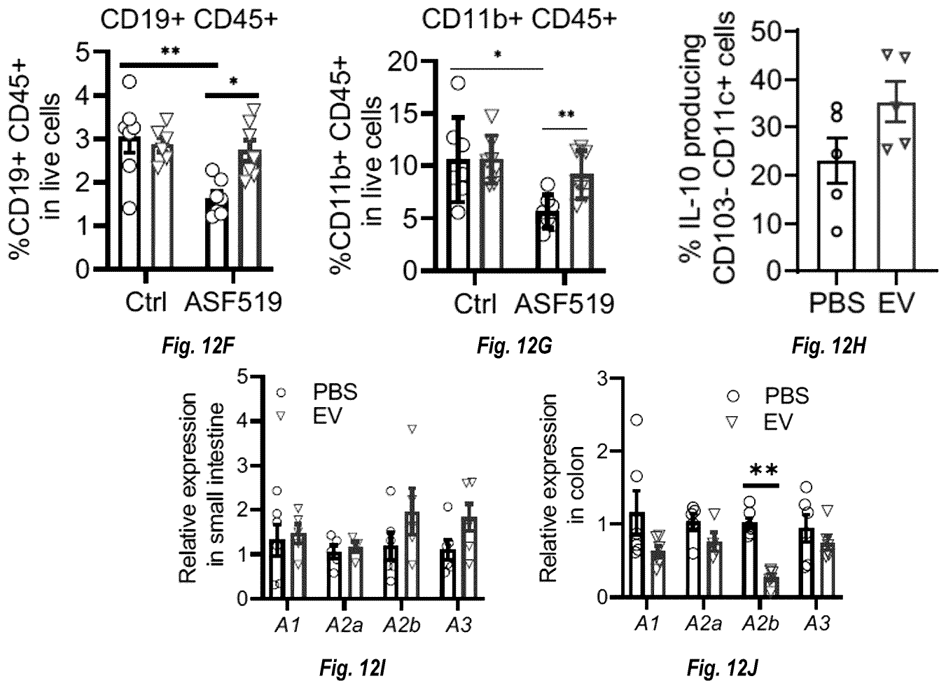

To test the involvement of adenosine receptors, adenosine receptor-specific antagonists were evaluated in BMDC assays. Intriguingly, supplementation of the A2a inhibitor ZM241385 drastically dampened IL-10 induction in a dose-dependent manner in response to ASF519 SSM (FIG. 2D, FIG. 12A). By contrast, antagonists of A1, A2b, and A3 at the tested concentrations had no significant impact. The engagement of A2a was further confirmed with the genetic knockout of A2a where A2a−/−BMDCs disabled IL-10 induction when exposed to ASF519 SSM extracts (FIG. 2E). A2a activation then increased the intracellular level of cyclic AMP (FIG. 12B), which is known to be involved in the transcriptional regulation of Il10. Therefore, activation of adenosine receptor A2a signaling in BMDCs is required for IL-10 induction.

To validate the role of adenosine receptor A2a in ASF519-mediated immunoregulation in vivo, A2a signaling was blocked by intraperitoneal injection of an A2a antagonist, ZM 241485, into antibiotic-pretreated mice that received live or heat-killed ASF519 (FIG. 2C). Immunophenotyping of colonic lamina propria (cLP) cells revealed an increase in IL-10 producing DCs among CD103− CD11c+ and CD103+ CD11c+ populations in live versus killed ASF519 recipients (FIG. 2D and 2E). Importantly, these effects were significantly decreased by the A2a antagonist. This IL-10 immunophenotype is consistent with the in vitro observation that blockage of A2a rather than other adenosine receptors sufficiently interrupted IL-10 production by BMDCs in response to ASF519 SSM. Other major immune cell types, such as CD11b+ F4/80+ macrophages, CD4+ T cells, and CD19+ CD45+ B cells, did not show significant changes in their IL-10 secretion (FIG. 12C-12E). It is also notable that ASF519-treated mice exhibited decreased lamina propria B cells and CD11b+ CD45+ cells in the colon (FIG. 12F and 12G), which was also restored by A2a blockage. Together, these data demonstrate that the immunoregulatory function of ASF519 in colonic lamina propria goes beyond IL-10 induction in DCs to include more general homeostatic activities of leukocytes through A2a activation.

### Example 5. Bacterial Extracellular Vesicles Induce IL-10 in BMDCs

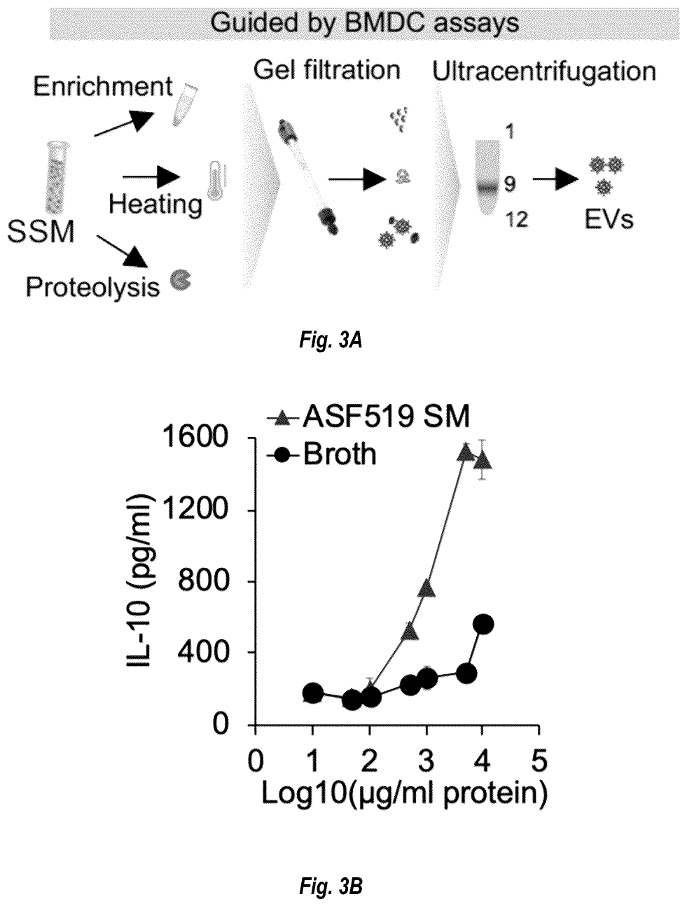

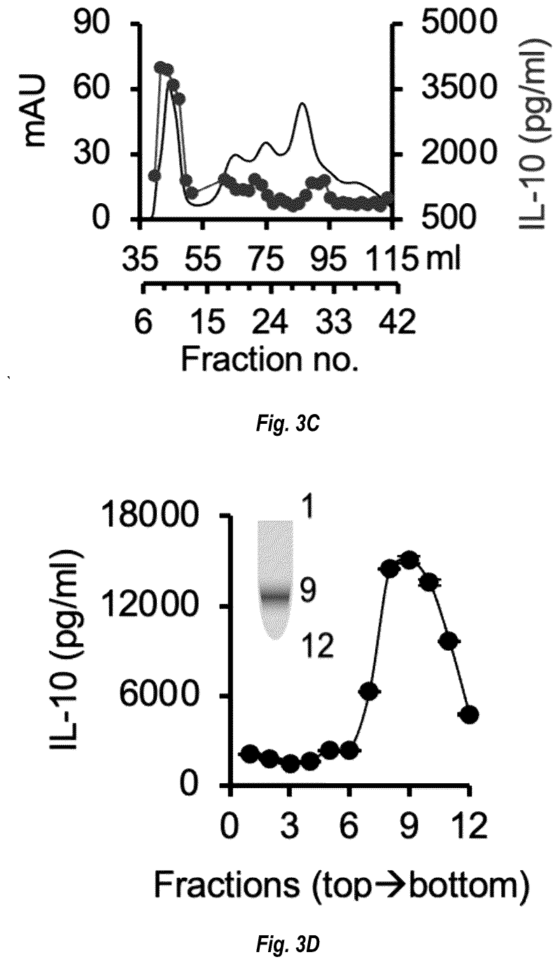

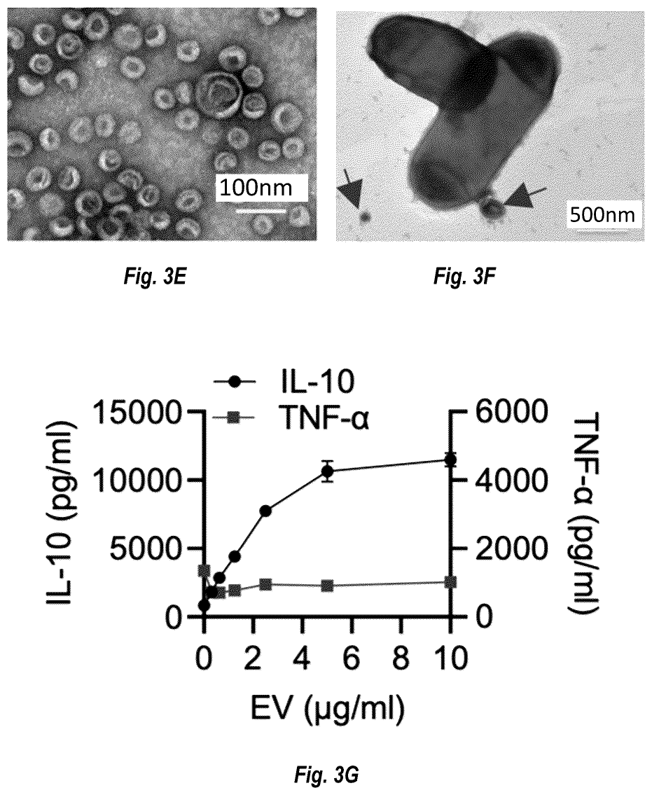

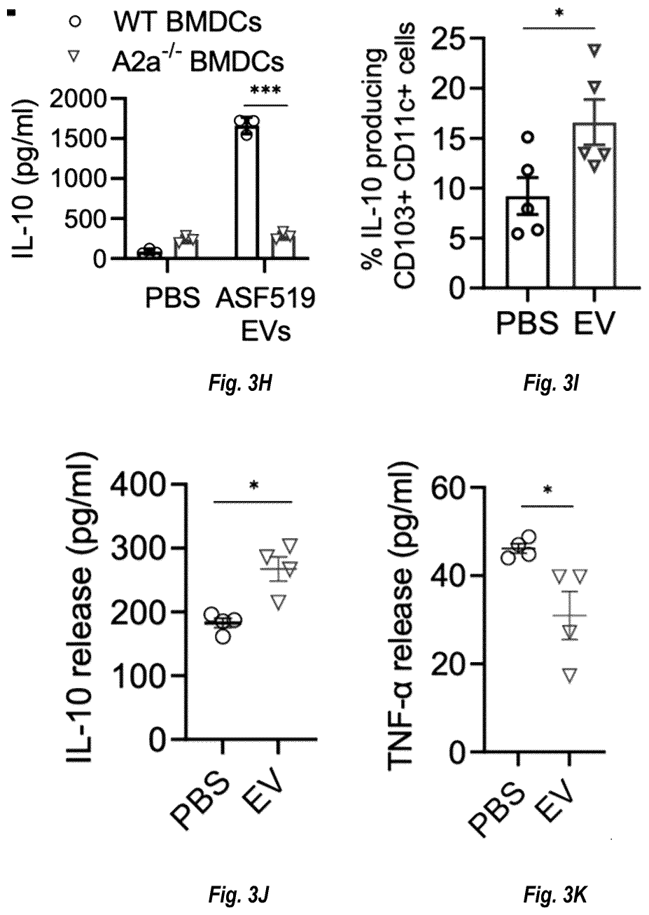

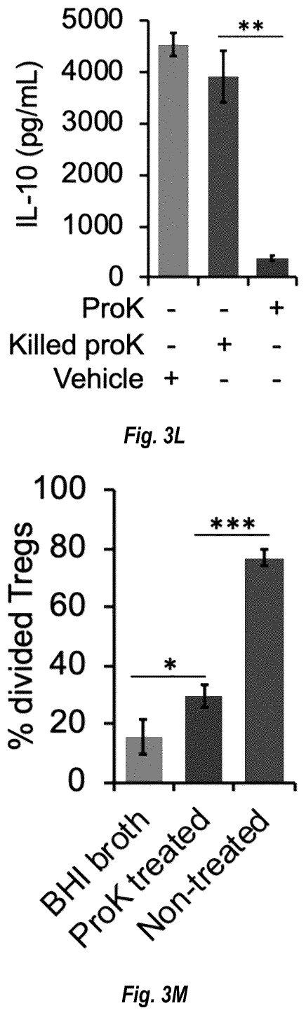

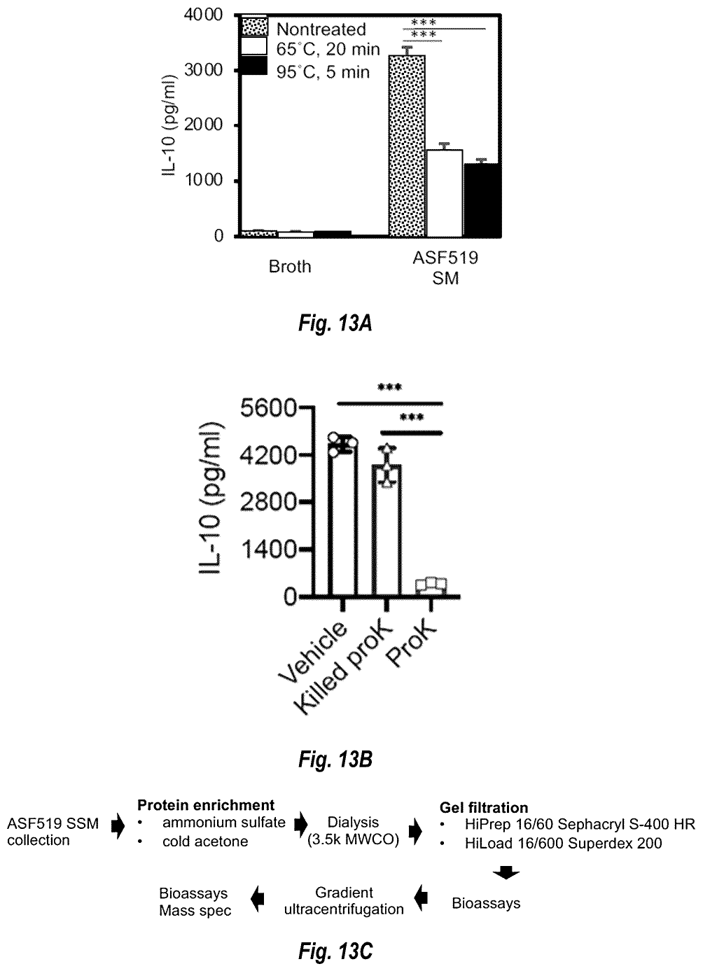

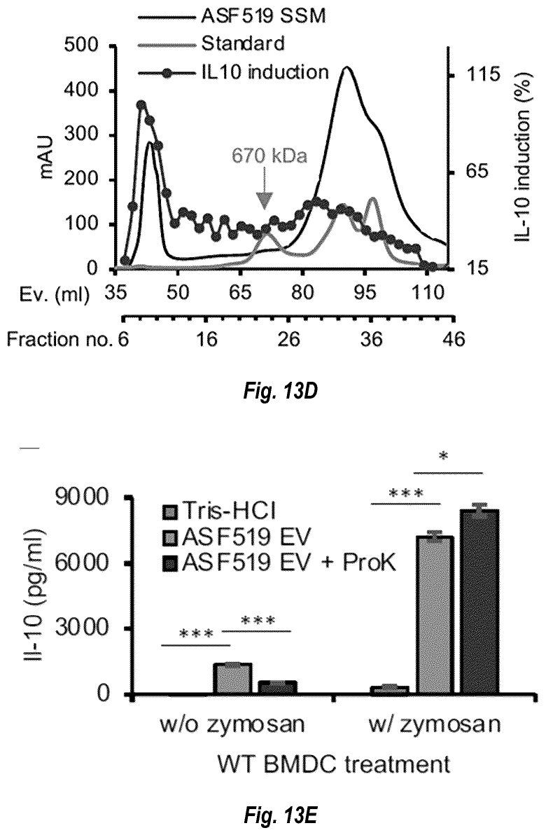

The potential responsible factors secreted from ASF519 that induce IL-10 and adenosine signaling were evaluated using bioanalytical techniques (FIG. 3A). The IL-10 inducing function of ASF519 SSM was largely heat-labile (FIG. 13A), and immobilized proteinase K digestion of ASF519 SSM largely abolished IL-10 induction (FIG. 13B). These results, plus a dose-dependent IL-10 induction by concentrated crude proteins (FIG. 3B), confirmed the proteinaceous nature of the responsible molecules. Gel filtration to fractionate crude proteins in ASF519 SSM was next performed (FIG. 13C). BMDC assays revealed consecutive fractions of an early elution peak exhibiting strong IL-10 induction (FIG. 3C). Protease K treatment (but not vehicle or heat-killed protease K) abolished IL-10 induction in BMDCs (FIG. 3L) and Treg expansion in BMDC-splenocyte co-coculture (FIG. 3M).

The elution chromatogram demonstrated that these bioactive fractions were in the excluded volume of the gel filtration column that was designed for separating 20-8,000 kDa globular proteins (FIG. 13B-13D), suggesting a large structure is formed. With density gradient ultracentrifugation of these bioactive fractions, the IL-10 inducing fractions (FIG. 3D) were captured and identified the ultrastructure to be bacterial extracellular vesicles (EVs) with high purity (FIG. 3E). Scanning electron microscopy then identified the formation of EVs during the growth of ASF519 on agar plates (FIG. 3F). These high-purity EVs were able to recapitulate a dose-dependent IL-10 induction in BMDC assays as observed with crude extracts of the bacterial spent medium, with a reduction in TNF-α production (FIG. 3G). The essential role of A2a in EV-mediated IL-10 induction in A2a^{-/-} BMDCs was also confirmed (FIG. 3H). These strong tolerogenic effects are distinct from the largely pro-inflammatory functions of previously characterized EVs from Pseudomonas aeruginosa (23), Salmonella typhimurium (24), Helicobacter pylori (25), Escherichia coli (26). In addition, immobilized proteinase K treatment of purified EVs diminished IL-10 production by EV-treated BMDCs (FIG. 8E), suggesting that proteinaceous components carried by EVs might be involved in IL-10 induction. These results together demonstrate the role of ASF519-derived EVs in IL-10 induction, which is partly mediated by undetermined EV components that are susceptible to proteolysis.

### Example 6. EV-Mediated IL-10 Production is Dependent on Autophagy

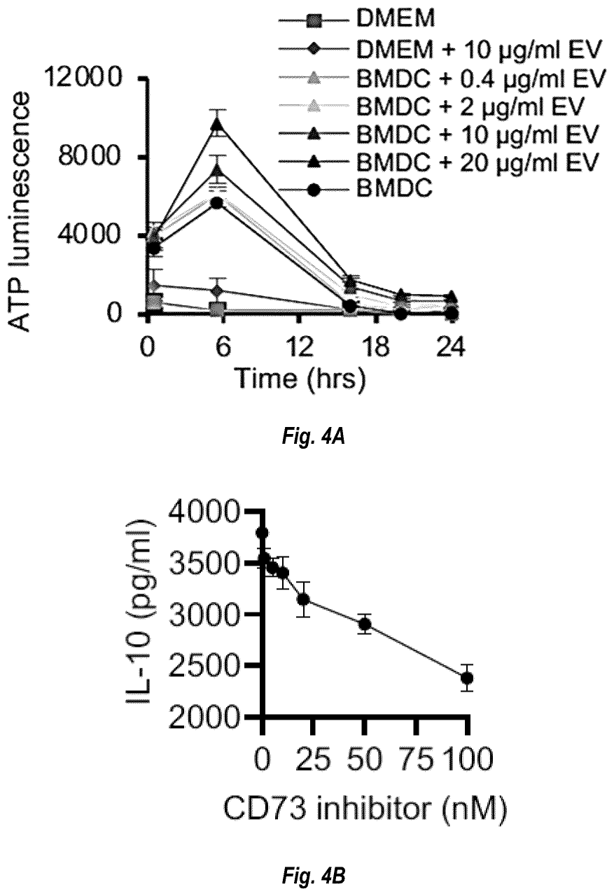

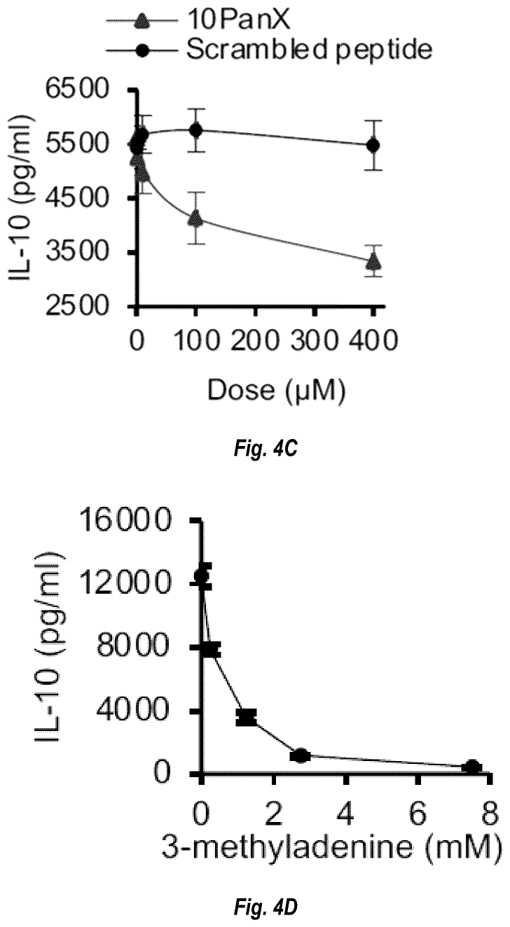

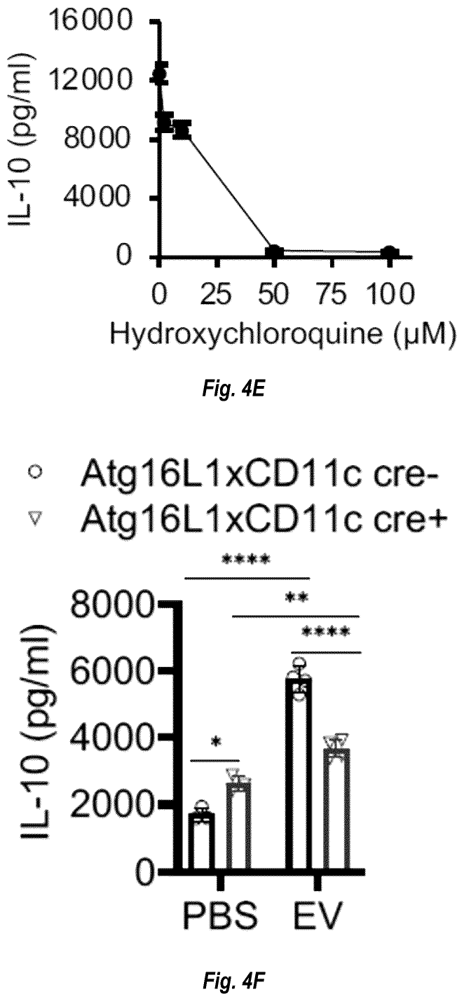

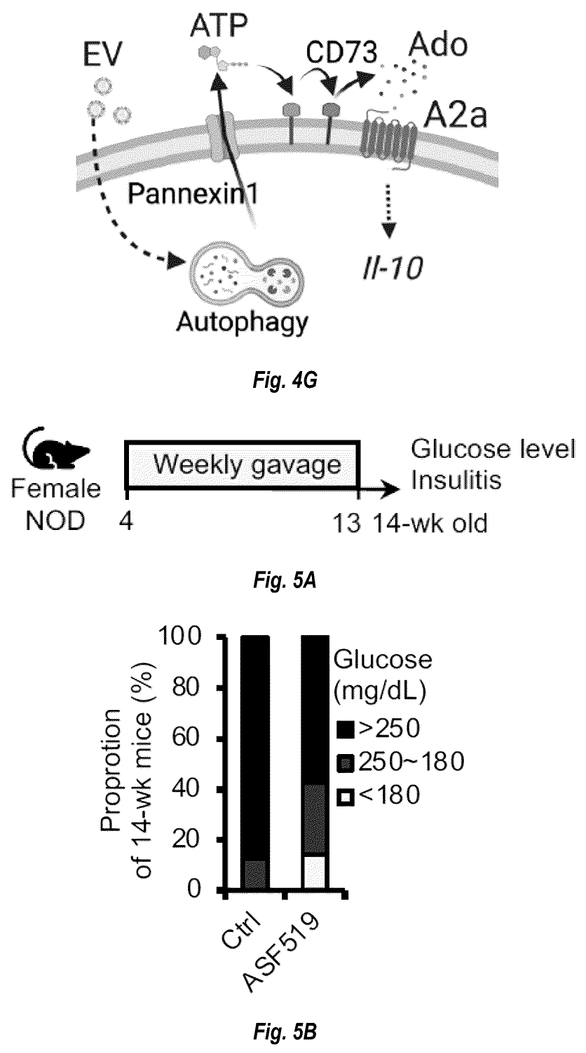

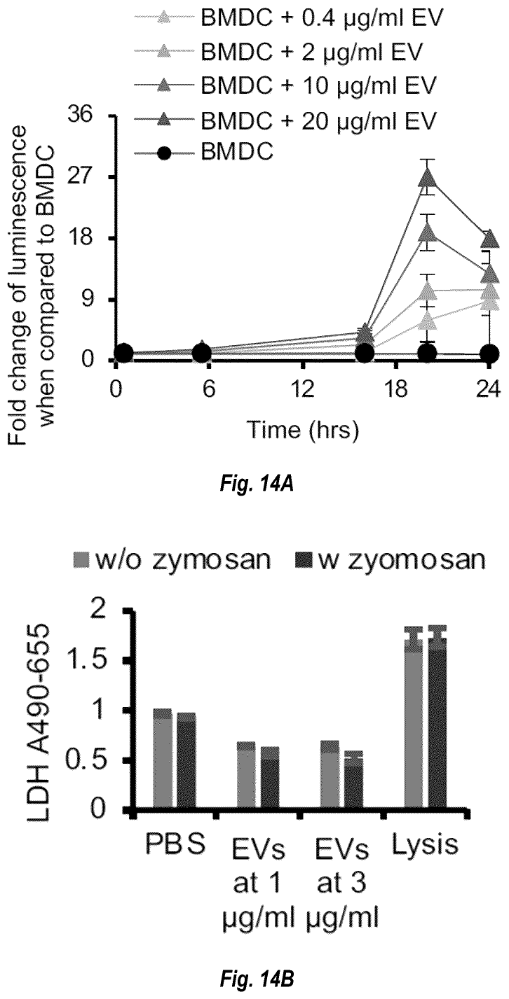

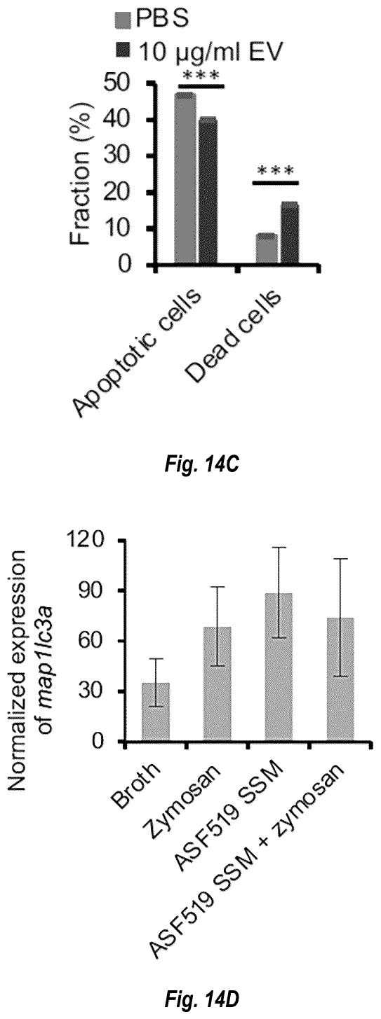

Adenosine can be generated by the sequential hydrolysis of extracellular ATP by ecto-nucleotidases CD39 and CD73 that are present on a variety of cell types. An increased level of extracellular ATP was seen in treated BMDCs, which positively correlates with EV dosage applied in assays (FIG. 4A, FIG. 14A). Pharmaceutical inhibition of adenosine-producing CD73 by PSB-12379 partly blocked IL-10 induction (FIG. 4B), suggesting the involvement of extracellular ATP hydrolysis. It is known that damaged membrane integrity, apoptosis, and autophagy cause ATP release, and the latter two mechanistically rely on membrane channels, such as pannexin-1 (29). Neither EV nor ASF519 SSM treatment enhanced the membrane permeability of BMDCs as indicated by the lactate dehydrogenase activity (FIG. 14B). Blockage of pannexin-1 dramatically dampened EV-dependent IL-10 induction (FIG. 4C); however, EV treatment did not promote apoptosis (FIG. 14C). Therefore, EV-induced ATP release, therefore, is unlikely through apoptosis.

To test the involvement of autophagy, pharmaceutical inhibition was performed at two stages of autophagy, autophagosome formation via the inhibition of class III PI3K with 3-methyladenine (3-MA) and fusion of autophagosomes with lysosomes using hydroxychloroquine (HCQ). Both inhibitors consistently and efficiently blocked IL-10 induction of EV-treated BMDCs (FIG. 4D and 4E). Moreover, genetic knockout of the Atg16L1 gene in Atg16L1×CD11c cre+ DCs, which is required for canonical autophagy, largely reduced IL-10 induction in response to EV stimulation (FIG. 4F), when compared to Atg16L1×CD11c cre− DCs. These results together demonstrate the involvement of autophagy in EV-mediated IL-10 production partly through pannexin-1-dependent ATP release and CD73-mediated adenosine formation (FIG. 4G).

### Example 7. Purified EVs Recapitulate Tolerogenic Phenotypes in Mice

To test if ASF519 EVs comparably promote immune tolerogenic phenotypes in vivo, daily intraperitoneal injection of purified EVs were administered to 4-week-old mice for a course of two weeks of treatment. Immunophenotyping of cLP cells revealed that the population of IL-10 producing CD103+ CD11c+ cells was doubled in EV recipient mice compared to the PBS control mice (FIG. 3I); a similar trend was observed in IL-10 producing CD103-CD11c+ cells even though it is not statistically significant (FIG. 12H). These changes are consistent with tolerogenic phenotypes observed in ASF519 recipient mice. To further validate the overall immune tolerance, the response of bulk cLP cells to immunogenic zymosan was examined. Through cytokine measurements, a higher level of IL-10 and a lower level of TNF-α in the supernatant of exposed cLP cells were seen after extraction from EV-treated mice (FIG. 3J and 3K). Both changes were not observed in the absence of zymosan during normal culturing. These data together suggest stronger peripheral tolerance to proinflammatory molecules is set by EV.

### Example 8. Oral Administration of ASF519 Ameliorates Autoimmune Disorders

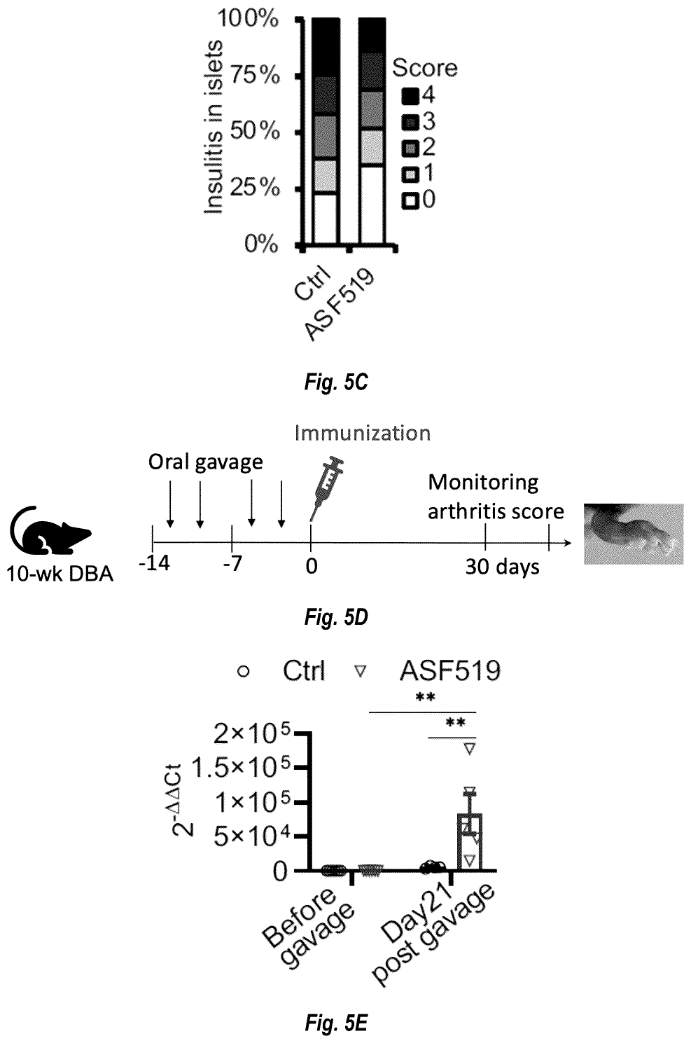

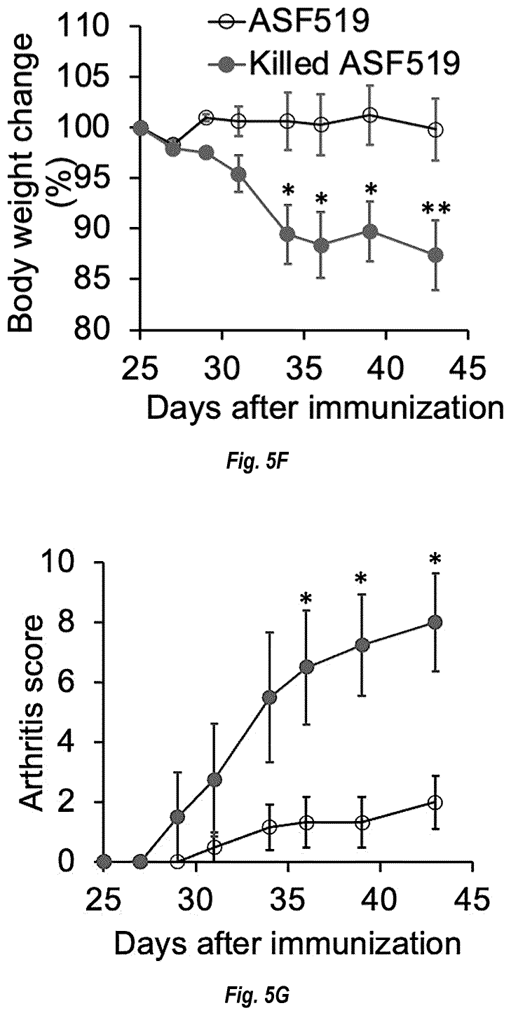

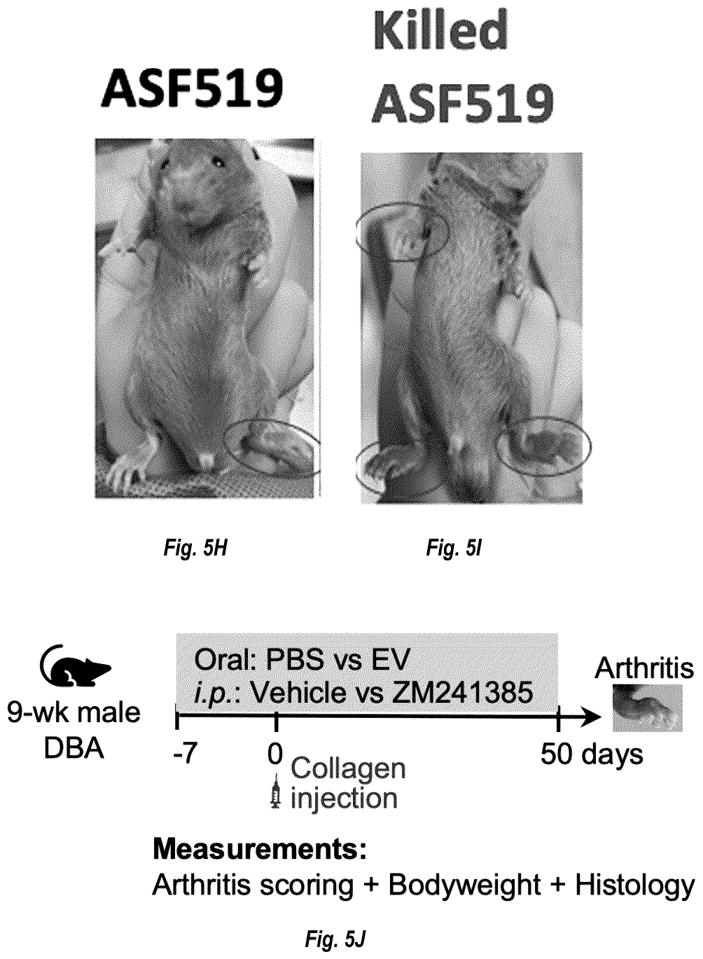

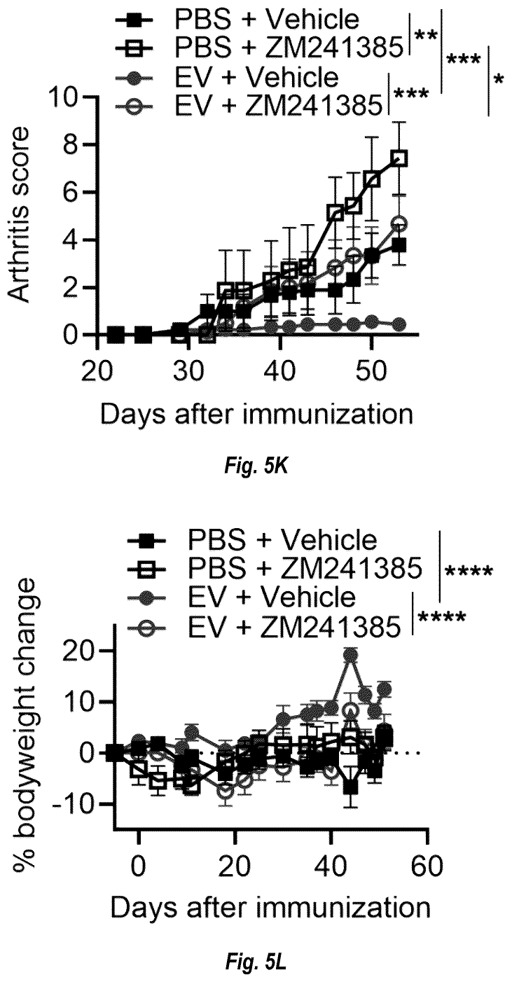

Type 1 diabetes (T1D) is an autoimmune disease in which insulin-producing beta cells in the pancreas are destroyed by T cells. DCs promote the priming and effector differentiation of autoreactive T cells in the pancreatic lymph nodes (30). To test if the immunoregulatory functions of ASF519 can curb the immunopathogenesis in T1D, ASF519 was administered to T1D-prone non-obese diabetic mice (NOD) by weekly oral gavage from four to thirteen weeks of age (FIG. 5A). At 14 weeks, ASF519 recipients had a lower incidence of hyperglycemia (>250 mg/dL, 57%) compared to killed ASF519 recipients (88%) (FIG. 5B), indicating an improved pancreatic function as a result of ASF519 supplementation. To measure immunopathogenesis directly in the pancreas, mice were sacrificed at 14-weeks of age to perform histological analysis of insulitis by scoring infiltration of insulin-producing beta cells by pathogenic lymphocytes. In line with improved glucose control, the severity of insulitis was reduced in ASF519 mice (FIG. 5C). Insulin immunostaining on adjacent sections of histological staining also demonstrated that more insulin-producing beta cells were present in the islets of ASF519 mice. Therefore, ASF519 colonization in NOD mice plays a protective role against T1D progression.

ASF519 was examined in other autoimmune disease models. Rheumatoid arthritis is an autoimmune disease that mainly involves the synovial tissues within joints. ASF519 was introduced to DBA mice before the injection of collagen to induce arthritis (FIG. 5D). The qPCR analysis confirmed the long-term colonization of ASF519 in the gut of DBA mice as after 3 weeks of the final gavage, the abundance of ASF519 in feces was >11 fold higher in live versus killed ASF519 recipient mice (FIG. 5E). Following 30 days post-collagen immunization, mice that received killed ASF519 significantly lost body weight (FIG. 5F), whereas ASF519 mice were able to maintain their body weight. In line with that, killed ASF519 recipient mice developed much more severe arthritis than ASF519 mice (FIGS. 5G-5I). These data demonstrate that ASF519 colonization strongly protected DBA mice from collagen-induced arthritis.

To test if purified ASF519 EVs protect collagen-induced arthritis through A2a signaling, 9-wk old male DBA/1J mice were orally administered with 200 μl of purified EVs (5 μg) in PBS for three times a week starting from one week before collagen immunization. At the time of EV administration, the inhibitor of the A2a receptor, ZM241385, was intraperitoneally injected with a volume of 100 μl (125 μg); the same volume of vehicle, consisting of 7.4% DMSO, 7.4% cremophor, and 85% PBS, was i.p. injected as the blockage control. Mice were immunized by subcutaneous injection of collagen as mentioned above. Mice were monitored two or three times a week for arthritis and bodyweight, and assessed arthritis severity by following the same protocol as mentioned above. Results in FIGS. 5J-5L show efficacy of EVs prepared from ASF519 in a collagen-induced arthritis model.

Accumulating evidence from mouse and human studies demonstrates that the microbiome is integral to immune regulation, and that alterations to the microbiome may underlie many immune-mediated and metabolic diseases. Here, distinct immunomodulatory functions of the ASF, an important and long-studied “model microbiome,” were studied. One specific member, P. goldsteinii ASF519, was a potent inducer of IL-10 in myeloid cells in an adenosine receptor A2a-dependent manner both in vitro and in vivo. ASF519 increases the availability of adenosine in the host through autophagy-dependent ATP hydrolysis by the ecto-nucleosidase CD73. EVs produced during ASF519 growth are responsible for A2a-dependent tolerogenic phenotypes, including IL-10 induction and TNF-a reduction, distinct from proinflammatory responses usually elicited by most bacterial EVs. Preceding work demonstrated that EVs from Bacteroides fragilis induce Treg IL-10 production, which could be disrupted by ATG16L1 knock-out in DCs (31-33 and US 2011/0251156), though how this regulated tolerance or IL-10 was unknown. Though the Parabacteroides belong to a distinct bacterial family (Tannerellaceae), ASF519 may share overlapping mechanisms of tolerance induction with B. fragilis. This study illustrates that the induction of IL-10 on myeloid cells by ASF519 EVs is mediated by A2a signaling via autophagy-dependent ATP release, directly linking autophagy of bacterial EVs to the production of tolerogenic IL-10. This study also broadens known microbiota-host receptor interactions to include adenosine A2a (34, 35). Future questions of interest include the nature of the proteolysis-sensitive EV components that induce IL-10, which is likely unrelated to the capsular polysaccharides identified on B. fragilis EVs (31-33). Also of interest is whether ASF519 EV-mediated A2a activation could have distinct biological functions in other cell types and tissues, given the central role of adenosine signaling to many aspects of mammalian physiology (36, 37).

This work serves as a model workflow by which future immune phenotypes can be screened in the ASF system in vitro and then interrogated in vivo towards the discovery and characterization of novel immunoregulatory functions of the gut microbiome. Furthermore, this workflow may inform combinatorial strategies to sharpen the regulatory potency of microbes or their secreted products for therapeutic use.

### Example 9. Materials and Methods for Examples 1-8

### Animal Models

C57BL/6, 129S1/J, Il10−/−, A2a−/−, DBA/1J, NOD mice were purchased from Jackson Laboratories and maintained in specific pathogen-free conditions. The A2a-/- mice were on a mixed genetic background of BALB/c and 129S. Atg16L1×CD11c cre+ and Atg16L1×CD11c cre− mice were kindly provided by Ramnick Xavier at Massachusetts General Hospital. Unless otherwise indicated, 8-12-week-old mice of both genders were used for in vitro experiments. Only male DBA and female NOD mice were used for disease studies. This study complied with all ethical regulations involving experiments with mice, and all experimental manipulations were performed in accordance with protocols approved by the Animal Care and Use Committee of Joslin Diabetes Center.

To study the in vivo function of Parabacteroides goldsteinii ASF519 in mice, this strain was introduced into 4-wk old both male and female germ-free (GF) mice by oral gavage of 200 μl bacterial cells with 1×10\hat{ }9 CFU in PBS. The control GF group was orally administered with 200 μl PBS. The SPF mice were developed by introducing fecal microbiota of conventional mice to GF mice. After 2 weeks of colonization, mice were sacrificed to collect sera, intestinal tissues, and cecal contents. Shotgun metagenomic sequencing for fecal and cecal samples confirmed mono-colonization. Approximately, 1 cm of middle colon and ileum (5 cm upstream of the cecum) were preserved in 0.5 ml TRIZOL buffer for storage at −80° C.

To examine the in vivo function of purified EVs, daily i.p. injection of EVs versus PBS to 4-wk old B6 males (10 μg high-purity EV per 100 μl per mouse) were performed. After a course of two weeks of treatment, cells were extracted from colonic lamina propria for immunophenotyping.

The engagement of adenosine receptor A2a was tested in ASF519-mediated immune regulation through pharmacological blockade in mice. 9-11-wk old C57BL/6J mice were treated by an antibiotic cocktail (0.5 g/L vancomycin, 1.36 g/L neomycin, plus 3.75 g/L sweeter aspartame) for 48 hrs. Then, the antibiotic water was exchanged with a 10% (w/w) PEG4000 solution in water, and the mice were fasted for 16 hours. After that, these prepared mice were dosing twice at the interval of 3 days by gavaging 200 μl of live or heat-killed P. goldsteinii in PBS at 1×10\hat{ }9 CFU. Both live and killed P. goldsteinii recipient groups were further divided with a daily intraperitoneal injection of 100 μl A2a antagonist ZM 241385 (1.25 mg/ml) or vehicle (7.4% DMSO+7.4% Cremophor+85.2% PBS) for 17 days. Stool samples were collected at 5 timepoints: before-antibiotic treatment, post-antibiotic/pre- 10% PEG4000 treatment, post-gavage day 1, day 3, and day 7. On post-gavage day 14, colon tissues and cecal contents were harvested for immunophenotyping and metabolomic analysis, respectively.

To test whether P. goldsteinii influences the development of autoimmune diabetes, this bacterium was introduced into non-obese diabetic mice (NOD), which are prone to develop insulitis and diabetes (38). 4-week-old female NOD mice received 200 μl of live or heat-killed ASF519 in PBS at approximately 1×10\hat{ }9 CFU by weekly oral administration until 13-weeks-old. Blood glucose was measured in tail tip blood at 14 weeks. The blood glucose at >250 mg/dL defines hyperglycemia (39). The pancreas was evaluated in histological studies, including Hematoxylin and Eosin staining and insulin immunostaining.

The protective role of P. goldsteinii in the collagen-induced arthritis mouse model (40), which is the most commonly studied autoimmune model of rheumatoid arthritis. 10-wk old male DBA/1J mice were orally administered with 200 μl of live or heat-killed ASF519 in PBS at 1×10\hat{ }9 CFU for twice a week in a course of two weeks. After microbial colonization, mice were immunized by 50 μl of 1 mg/ml collagen emulsion in Complete Freund's Adjuvant (CFA, Sigma-Aldrich #F5506-10ML) that consists of 1 mg/ml of heat-killed Mycobacterium tuberculosis (Invitrogen #tlrl-hkmt-1) by subcutaneous injection at about 1.5 cm distal from the base of the tail. This formulation in CFA has been demonstrated to be essential to induce arthritis in mice from the autoimmune response that results in arthritis of major joints within 4-6 weeks of injection. Mice were monitored twice weekly for arthritis and bodyweight, assessing arthritis severity by the established qualitative scoring system through observing and measuring the severity of paw inflammation (40). Each paw was evaluated and scored individually on a scale of 0-4, with 4 indicating the most severe inflammation. The maximal combined score from 4 paws per mouse is 16.

To test if purified ASF519 EVs protect collagen-induced arthritis through A2a signaling, 9-wk old male DBA/1J mice were orally administered with 200 μl of purified EVs (5 μg) in PBS for three times a week starting from one week before collagen immunization. At the time of EV administration, the inhibitor of the A2a receptor, ZM241385, was intraperitoneally injected with a volume of 100 μl (125 μg); the same volume of vehicle, consisting of 7.4% DMSO, 7.4% cremophor, and 85% PBS, was i.p. injected as the blockage control. Mice were immunized by subcutaneous injection of collagen as mentioned above. Mice were monitored two or three times a week for arthritis and bodyweight, and arthritis severity was assessed by following the same protocol as mentioned above for ASF519 treatment. Finally, mice were sacrificed to collect tissue samples for histology and other measurements.

### Collection of Bacterial Spent Media

ASF isolates were provided by Dr. Michael Wannemuehler at Iowa State University. Each ASF member was grown in the brain heart infusion broth (BHI) under the anaerobic condition at 37° C. The BHI medium consists of 37 g/L BHI (Sigma), 5 g/L yeast extract, 0.5 g/L L-cysteine, 10 mg/L resazurin, and 5 mg/L hemin, dissolved in ½ (v/v) miniQ water and ½ (v/v) tap water. After autoclaving, 1 L of BHI was supplemented with 0.2 ml 1% vitamin K, 50 ml newborn calf serum, and 50 ml horse serum, and 10 ml of 20% (m/v) filter-sterilized yeast extract. The medium was placed in the anaerobic chamber overnight to reduce oxygen before bacterial inoculation. The spent media were harvested at indicated time points during growth and then filter-sterilized twice with 0.2 μM filters before storage at −80° C. Bacterial cell biomass was estimated by monitoring OD600.

The defined medium used to grow ASF519 contains salts, carbon sources, and supplements. The salts and carbon sources include 13.6 g/L KH2PO4, 0.875 g/L NaCl, 1.125 g/L (NH4) 2SO4, 5.0 g/L glucose, and 0.25 g/L L-cysteine, pH7.2. 1 L of the salts/carbon sources was then supplemented with 0.5 ml of 10 mg/ml hemin, 200 μl of 1% vitamin K, 1.0 ml of 0.1 M MgCl₂, 1.0 ml of 0.4 mg/ml FeSO₄7H₂O, 1.0 ml of 0.8% CaCl₂, 10 ml ATCC Vitamin complex, and 10 ml ATCC mineral complex. The medium was sterilized with 0.2 μm filters and oxygen-reduced in an anaerobic chamber before use.

Alternatively, isolates of bacteria comprised in Altered Schaedler Flora (ASF), such as P. goldsteinii, could prepared from fecal pellets of gnotobiotic animals that are colonized exclusively with Altered Schaedler Flora (ASF, available from Taconic, Item No. ASF-DONOR-M or ASF-DONOR-F). These pellets could be cultured under conditions on FASTIDIOUS ANAEROBE BROTH (DSMZ Medium 1203a) to obtain colonies of the presently described P. goldsteinii strain.

### Cell Isolation and Cultivation

Bone marrow-derived dendritic cells (BMDCs) were developed from mouse bone marrow. Mice at 8-12 weeks old were euthanized and sacrificed to collect hind legs. After removing skin, flesh, and muscles from the legs, bone marrow was flushed out from bones and collected for red blood cell lysis using the RBC lysis buffer (Invitrogen #00-4333-57) by following the manufacturer's instruction. After centrifugation at 500 g for 8 min at 4° C., hematopoietic cells were suspended in complete DMEM (cDMEM) consisting of 10% fetal calf serum, 1× penicillin-streptomycin solution (100×, Caisson Labs #PSL01), 10 mM HEPES, and 5 μM 2-mercaptoethanol in Gibco DMEM medium (Thermo fisher #10-566-024). To develop BMDCs, hematopoietic cells were cultured in cDMEM with 20 ng/ml GM-CSF (2×106 cells in 10 ml per untreated petri dish 100 mm×15 mm) at 37° C., 5% CO₂. The supplemented medium was refreshed on days 3 and 6. BMDCs were collected on day 9 and resuspended in the CryoStor CS10 buffer (STEMCELL #07930) for storage at −80° C. Following the same protocol, Atg16L1×CD11c cre+ and cre− BMDCs were cultivated from hind legs of Atg16L1×CD11c cre− or cre+ mice provided by the Xavier lab. DCs were enriched from BMDCs with EasySep mouse pan-DC enrichment kit (STEMCELL, #19763) and then used for EV treatment.

To develop BM-DMs (41), hematopoietic cells from bones were transferred into culture-treated dishes (on average 1 leg per plate) for incubation at 37° C. for 4 hours to allow resident macrophage to adhere to the plastic. Cells in the supernatants were collected and cultured in cDMEM with 10 ng/ml M-CSF (2×106 cells in 10 ml per culture treated dish 100 mm×15 mm) at 37° C., 5% CO₂, and the medium was refreshed every 3 days. BM-DMs were collected on day 10 for downstream experiments.

The BEND3 endothelial cell line was cultured in cDMEM without using GM-CSF or M-CSF (5×105 cells in 10 ml per culture treated dish 100 mm×15 mm) at 37° C., 5% CO2. After 3 days, the medium was removed; cells were treated with 0.25% trypsin (3 ml per petri dish) at 37° C. for 5 min to allow cell dissociation. Then, the cells were gently scraped from the plastic and resuspended cells in fresh cDMEM for further assays.

PanB, panT, panDC, and monocytes were isolated from splenocytes of 8-12 wk B6 mice using EasySep Mouse B cell isolation kit (STEMCELL #19854), EasySep Mouse T cell isolation kit (STEMCELL #19848), EasySep Mouse Pan-DC enrichment kit (STEMCELL #19763), and EasySep Mouse Monocyte isolation kit (STEMCELL #19861), respectively.

### In Vitro Stimulation

1×10⁵ cultured or isolated immune cells in 140 μ1 cDMEM were placed in a 96-well non-tissue culture treated microplate (Corning #351172), mixed with 60 μl of filter-sterilized spent media (SSMs) for 48 hours incubation at 37° C., 5% CO₂, then supplemented with zymosan at 4 μg/ml for additional 16 hours incubation under the same condition. Finally, stimulated cultures were centrifuged at 500 g for 8 min to harvest supernatants for the measurement of cytokines. To test the effect of ASF519 SSMs on TGF-β production, no zymosan was added during the entire course (64 hours) of stimulation of BMDCs. IL-10, TGF-β, and TNF-α were quantified by corresponding ELISA kits (Invitrogen).

The impact on Treg proliferation was tested by co-culturing stimulated BMDCs with splenocytes. 1×10⁶ BMDCs were placed in 1 ml cDMEM into a 24-well non-tissue culture treated plate (Corning #351147), stimulated cells with 0.5 ml of bacterial SSMs or BHI broth for 2 days at 37° C. in the CO2 chamber, then further stimulated cells with 4 μg/ml zymosan for additional 16 hours under the same condition. Fresh splenocytes from 129S1 mice were diluted to 1×10\hat{ }6 cells/ml in phosphate-buffered saline (PBS), stained with Celltrace violet (Thermo fisher #C34571) at 10 μM, 37° C. for 20 min, washed once with five volumes of Gibco RPMI1640 medium (Thermo fisher #61870127), and finally resuspended in RPMI1640 at 2×10\hat{ }6 cell/ml. To coculture BMDCs and splenocytes, the supernatants were removed from stimulated BMDCs by centrifugation at 500 g for 8 minutes, cell pellets resuspended in 0.5 ml RPMI1640, and then 0.5 ml of stained splenocytes added for 4 days incubation in the CO2 incubator at 37° C. Treg proliferation was analyzed by flow cytometry for co-cultured cells stained by mouse CD4 and Foxp3 antibodies.

To understand the immunological properties of colonic lamina propria (cLP) cells extracted from EV or PBS treated mice, the response of heterogenous cLP cells was tested to immunogenic zymosan. Each sample has two reactions; each reaction has 9.5×10\hat{ }5 extracted cells in 800 μl supplemented DMEM in a 24-well plate. Cells were then incubated at 37° C. for 2 hrs in the CO2 chamber. One set of reactions was subjected to zymosan stimulation at 4 μg/ml; the other set was treated with the same volume of PBS as zymosan supplementation. After 24 hrs treatment in the CO₂ chamber, supernatants were collected for the measurement of IL-10 and TNF-a with ELISA kits.

### Flow Cytometry

Single-cell suspensions were generated from mouse colonic lamina propria tissue using the Lamina Propria dissociation kit (Miltenyi Biotec #130-097-410) by following manufacturers' instructions. The program 37° C._m_LPDK_1 on the gentleMACS Octo dissociator with heaters was used for tissue dissociation and cell extraction. Spleens were harvested and gently grinded with the rubber head of syringe plunge through 70 μm cell strainers to obtain signal cell suspension; red blood cells were then lysed with the RBC lysis buffer (Invitrogen #00-4333-57). Cells were preserved in the CryoStor CS10 buffer (STEMCELL #07930) for storage at −80° C.

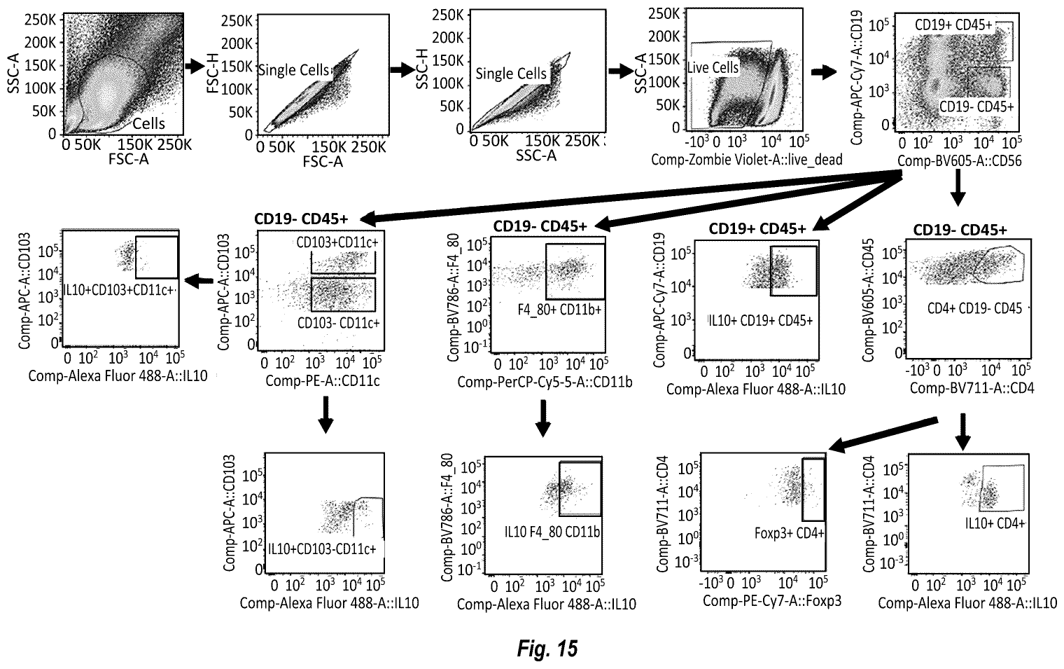

Single-cell suspensions were stained and analyzed with BD LSRFortessa. The panel included fluorescence conjugated antibodies against CD45, CD4, CD19, CD11b, CD11c, CD103, F4/80, Foxp3, and IL-10. Cells were first stained by the Zombi Violet Fixable Viability Kit, incubated with the FcR blocking reagent (anti-CD16/CD32, BioLegend #101302) in the cell staining buffer (BioLegend #420201) at room temperature for 10 minutes, and then stained for surface markers, including CD45, CD4, CD19, CD11b, CD11c, CD103, and F4/80. Stained cells were then fixed and permeabilized by using the Foxp3 Fix/Perm buffer kit (BioLegend #421403). Permeabilized cells were finally stained for intracellular and nucleus markers. Cells were acquired with a BD LSRFortessa, and analysis was performed with the FlowJo software. Frequencies of each cell type were averaged for each experimental condition. Gating conditions are shown in FIG. 15.

For flow cytometry analysis of co-cultured cells, the panel included fluorescence conjugated antibodies against CD4, CD8, and Foxp3. Cells were stained with the APC eFluo 780 live/dead dye and then corresponding antibodies by following the same staining protocol used for colonic lamina propria cells.

To test the effect of EV treatment on apoptosis, 2×10\hat{ }6 BMDCs in 1 ml supplemented DMEM were seeded into 24-w plates, then treated with 10 μg/ml purified EVs. After 48 hrs incubation in the CO₂ chamber, zymosan was added at 4 μg/ml for an additional 16-hour stimulation. BMDCs were collected for staining with the dead cell apoptosis kit with annexin V FITC and PI (Thermo scientific #V13242) and analyzed samples with a BD LSRFortessa.

### Low Input RNA-Seq

1,000 BMDC cells were collected directly into 5 μl TCL lysis buffer (Qiagen) with 1% 2-mercaptoethanol. Lysates were frozen after 5 min. Smart-seq2 libraries were prepared as previously described (42) and sequenced using the Broad Genomics Platform. Reads were aligned to the mouse genome (GENCODE GRCm38/mm 10 primary assembly and gene annotation version M16; www.gencodegenes.org/mouse/release_M16). The ribosomal RNA gene annotations were removed from the general transfer format (GTF) file. The gene-level quantification was calculated by featureCounts (www.subread.sourceforge.net/). Raw read count tables were normalized by the median of ratios method with the DESeq2 package from

Bioconductor and then converted to GenePattern GCT and CLS format. Samples with less than 3 million uniquely mapped reads were automatically excluded from normalization to mitigate the effect of samples with poor quality on normalized counts. Normalized read counts were filtered for robust expression (>10) to avoid confounders from low-level noise and processed in the Multiplot suite and Morpheus (https://software.broadinstitute.org/morpheus/). PCA was done using the prcomp function in R on all genes.

### Metagenomic Data Analysis

The genomic DNA was extracted from feces or cecal contents using the ZymoBIOMICS DNA kit (Zymo #D6300). Shotgun metagenomic sequencing was performed using Illumina HiSeq2000 NextGen Sequencer. Data was cleaned by removing mouse genome contamination. Microbial abundance was analyzed by MetaPhlAn2 (43). The prevalence of Parabacteroides goldsteinii in the human gut microbiome studies was analyzed by the curatedMetagenomicData package (44) in R.

### Bioassays for Treated or Fractionated Samples

To perform acetone precipitation, 4 times the sample volume of cold (−20° C.) acetone to ASF519 SSMs or medium broth in an acetone-compatible bottle were mixed and then incubated the mix for at least 60 min at −20° C. The precipitate was collected by centrifugation at 13,000g for 10 minutes, then air-dried at room temperature for 30 minutes before adding 1× PBS buffer to dissolve the protein pellet on ice. Protein concentration was measured by the Pierce BCA protein assay kit (Thermo Fisher #23227). The IL-10 inducing capability of acetone precipitates was evaluated at varying working concentrations in BMDC assays. To verify the role of proteinaceous components in IL-10 induction and Treg proliferation, SSMs of ASF519 were grown in either the defined medium or supplemented BHI with immobilized proteinase K to destroy proteins. In detail, 2 ml of 33 mM Tris-HCl pH7.5 was added to reconstitute 10 U protease-agarose power (1U/200 μl, Sigma #P9290-10UN), 1 ml protein sample with 200 μl of reconstituted protease was mixed, protease killing was performed (by heating at 95° C. for 10 minutes), or 33 mM Tris-HCl pH7.5, for incubation at 37° C. for 1 hour, followed by centrifugation to remove protease-agarose beads. By following the in vitro BMDC stimulation protocol described above, the IL-10 inducing capability of heat-and proteinase K treated samples was evaluated. The impact on Treg proliferation was tested by coculturing cell tracer labeled splenocytes with BMDCs that had been stimulated by proteinase K treated or non-treated samples.

For gel filtration to fractionate proteins by size, the concentrated crude samples were loaded, following filtration using 0.2 μm syringe filters, to the AKTA pure system with a 10 ml sample injection loop and a HiLoad 16/600 Superdex 200 pg column. Proteins were eluted into 1× PBS at 1 ml/min and collected every 2 ml in sterile microtubes. In vitro BMDC assays using 10 μl of each fraction as the stimulus were evaluated to determine bioactive fractions that can induce IL-10 production. The bioactive fractions were pooled and concentrated for further analysis, including protein quantification, native-/SDS-PAGE analysis, protein identification by mass spectrometry, and gradient ultracentrifugation. The gel filtration standard (Bio-Rad #1511901) was analyzed with the same gel filtration settings to indicate the molecular size.

To perform gradient ultracentrifugation, 2 ml of EVs containing 45% (m/v) iodixanol (STEMCell, #07820) was loaded to the bottom of ultra-clear tubes (Beckman coulter #344059), then sequentially topped with 2 ml of 35%, 30%, 25%, 20%, 10% iodixanol using PBS as a diluent. Ultracentrifugation was carried out at 4° C., 180,000 g for 3 hrs in Optima XE-90 ultracentrifuge. 1 ml fractions were manually prepared from top to bottom. Each fraction was subjected to buffer exchange with PBS using Amicon centrifugal filters (Fisher Scientific #UFC501024), followed by BMDC assays to identify IL-10 inducing fractions. Those bioactive fractions were concentrated and stored at −80° C.

Bioactive fractions and ASF519 colonies developed on BHI agar were negatively stained before imaging. BMDC assays with wildtype or A2a−/− genotype were performed as mentioned above to test cytokine production in response to the supplementation of purified EVs. For mouse experiment, daily i.p. injection of EVs versus PBS were administered to 4-wk old B6 males (10 μg high-purity EV per 100 μl per mouse). After a course of two weeks of treatment, cells were extracted from colonic lamina propria for immunophenotyping. Lamina propria cells were subjected to zymosan stimulation before collecting supernants for measurement of IL-10 and TNF-α.

### Pharmacological Inhibition

To test whether ASF519 mediated IL-10 induction occurs via any of the adenosine receptors, the AHCY inhibitor 3-deazeadenosine (DZA, Sigma #D8296-5MG) to block the formation of adenosine, the A1 antagonist DPCPX (Tocris #0439), the A2a antagonist ZM 241385 (Thermo fisher #10-361-0) and the A2b antagonist MRS-1754 (Tocris #2752), or the A3 antagonist MRS-1523 (Sigma #M1809-5 MG) were each evaluated. Stocks of antagonists were prepared in 50% DMSO. 1× 10⁵ BMDCs were treated in 140 μl with 2 μl of a series of diluted antagonists or vehicle (50% DMSO) at 37° C. for 30 minutes, then added 60 μl of broth control, ASF519 SSMs, or protein extracts from SSMs to stimulate BMDCs for 48 hours in the CO2 chamber, followed by further stimulation with zymosan at 4 μg/ml for 16 hrs at 37° C. in the CO2 chamber. Finally, the cell culture was centrifuged to collect supernatants for IL-10 ELISA assays.

To block autophagy in BMDC assays in response to purified EVs, 3-methyladenine (Tocris #3977) and wortmannin (Tocris #1232) to inhibit autophagosome formation and hydroxychloroquine (Tocris #5648) to inhibit the fusion of autophagosomes with lysosomes were each applied. The ecto-nucleosidase CD73 was blocked with PSB-12379 (Tocris #12379). Unless otherwise indicated, all inhibitors were reconstituted and diluted with supplemented DMEM. The ATP release channel, pannexin-1, was inhibited by 10PanX (Tocris #3348), using a scrambled peptide (Tocris #3708) as control. The working concentration of purified EVs was indicated specifically, gently mixed with 1×10\hat{ }5 BMDCs in 200 μl before adding these inhibitors. After 48 hours incubation in the CO₂ chamber, zymosan was added at 4 μg/ml for additional 16 hours stimulation. Supernatants of BMDCs were collected for IL-10 quantification.

The impact of TNF signaling on IL-10 induction of BMDCs was tested by blocking the TNF signaling pathway with Etanercept (45) (Sigma-Aldrich #Y0001969) in the BMDC assay using ASF519 SSMs and zymosan for step-wise stimulation. The levels of IL-10 and TNF-α in the BMDC supernatants were measured by ELISA kits (Invitrogen).

### Negative Staining of EVs

5 μl of EV sample was adsorbed for 1 min to a carbon coated grid (EMS, #CF400-CU) that had been made hydrophilic by a 20 sec exposure to a glow discharge (25 mA). Excess liquid was removed with filter paper (Whatman #1), the grid was then floated briefly on a drop of water, blotted again on a filter paper and then stained with 0.75% uranyl formate (EMS, #22451) for 15 sec. After removing the excess uranyl formate with a filter paper, the grids were examined in a JEOL 1200EX Transmission electron microscope, and images were recorded with an AMT 2k CCD camera. For bacteria grown on a plate, fixative (1% glutaraldehyde) was added to the colony, the grid was floated directly on top of the liquid for 2 min, and then 1% phosphotungtic acid (pH 7) was used instead of uranyl formate to stain cells.

### Histological Analyses

Hematoxylin and Eosin (H&E) staining was performed to examine infiltration in pancreatic islets. The slides were deparaffinized in two washes of xylenes at 5 min and then rehydrated in series of decreasing Ethanol concentration, 100%, 95%, 70%, and 50% and distilled water. The slides were stained with hematoxylin (Gill-3, Fisher #3537-32) for 2 min and raised in running distilled water. The slides were destained (3-4 dips) with acid water and raised with distilled water then blued with ammonia water. After washing with distilled water and a quick 95% ethanol wash, the slides were stained with eosin (Thermo Scientific #6766007) and dehydrated with two washes of 95% ethanol, two washes of 100% ethanol, and two washes of xylene. The slides were mounted with permount (Fisher #SP15-500), cover with a coverslip, and left to dry overnight. Images were taken with EVOS® FL Auto Imaging System (Life Technologies) at 20× in brightfield. The severity of insulitis in islets was assessed by bind pathological scoring.

To confirm the insulin producing function of islets, immunohistochemical staining of insulin in pancreas was performed. The slides adjacent to those used in H&E staining were deparaffinized in xylenes at 14 minutes and then rehydrated in series of decreasing ethanol concentration, 100% for 8 minutes, 95% for 6 minutes, and 70% for 10 minutes then in distilled water for 5 minutes. The slides were washed with PBS w/0.2% lamb serum for 5 min and then outlined with a PAP pen. The slides were blocked with Avidin (Vector Laboratories, #SP-2001) for 30 minutes, washed with PBS w/0.2% lamb serum for 5 min, and then blocked with Biotin (Vector Laboratories, #SP-2001) for 30 min and washed again with PBS w/0.2% lamb serum. The slides were blocked with goat serum (Vector Laboratories, #PK-4007) for 1 hour and incubated overnight at 4° C. with an anti-Insulin antibody (ab195956, 1:1000). The slide was warmed to room temperature for 1 hour and washed with PBS w/0.2% lamb serum for 5 min and then incubated with a biotinylated secondary antibody (Vector Laboratories, #PK-4007) for one hour and washed PBS w/0.2% lamb serum for 5 min. Then the slides were incubated with the ABC reagent (Vector Laboratories, #PK-4007) for 1 hour and washed with PBS. The slides were incubated in a peroxidase substrate solution (Vector Laboratories, #SK-4105) for 2 minutes and washed with distilled water and counterstained with hematoxylin (Gill-3, Fisher #3537-32) for 30 sec, and raised in running distilled water. The slides were then blued with ammonia water for 3 minutes and raised in running distilled waters. The slides were dehydrated with a series of washes with 70% ethanol for 5 minutes, 95% ethanol for 2 min, 100% ethanol for 2 min, and xylene for 4 min. The slides were mounted with permount (Fisher #SP15-500), covered with a coverslip, and left to dry overnight. Images were taken with EVOS® FL Auto Imaging System (Life Technologies) at 20× in brightfield. The islets without insulitis still had functional beta cells by immunostaining for insulin.

Mouse Magnetic Luminex assays

A magnetic bead-based multiplex assay (R&D systems #LXSAMSM-18) was performed for the measurement of 18 analytes in sera, including BAFF/BLyS, IFN-gamma, IL-1beta, IL-3, IL-5, IL-10, IL-13, IL-17E, IL-33, GM-CSF, IL-1alpha, IL-2, IL-4, IL-6, IL-12p70, IL-17A, IL-27, and TNF-alpha. Serum samples were analyzed with two replicates in a 96-w plate by following the manufacturer's instructions. The plate was read with Luminex 200.

### Metabolomics

Metabolites from approximately 50 mg cecal contents or a single fecal pellet were extracted for the analysis of untargeted metabolomics. In detail, cecal/fecal samples were suspended in 2 ml methanol in 8 ml glass vials (VWR #66011-085) and 4 ml cold chloroform was added for vortex mixing and homogenization in an ultrasound water bath for 5 minutes. 2 ml LC-MS grade water consisting of 100 nM pentanoic acid as the internal standard was added. After vortex mixing, vials were placed in centrifuge tubes for centrifugation at 3,000 rpm for 10 minutes. The aqueous phase was transferred to new glass vials and dried with nitrogen. Metabolites were resuspended in a volume of acetonitrile: water (1:1) based on the sample weight (200 μl/100 mg).

Samples were run on a Thermo Fisher Orbitrap IDX mass spectrometer. 5 μl of the extracted sample was injected onto a 5 μm Zic-PHILIC column (150×2.1 mm). Solvent A contained 20 mM ammonium carbonate and 0.1% ammonium hydroxide in Water; solvent B contained 97% acetonitrile in water. A linear gradient was used to separate the analytes: 100 to 40% solvent B (0 to 19 min), 40 to 0% solvent B (19 to 28 min), 0% solvent B (28 to 33 min), 0 to 100% solvent B (33 to 36 min), and re-equilibration of the column with 100% solvent B (36 to 45 min) at a flow rate of 0.2 ml/min and a column temperature of 40° C.

The mass spectrometry parameters were as follows: HESI+ and −; for MS1: switching polarity, 120,000 resolution, range 65-1000, 40% RF lens, AGC 1.e5, maximum IT 50 ms, using internal calibration; for MS2: threshold at 2e4, dynamic exclusion, AquirX targeted exclusion, AquirX targeted inclusion—ddMS2 with HCD stepped 15, 25, 45, detected in orbitrap at 30,000 resolution, cycle of 0.6 sec with MS2 of other if time, same parameters as the inclusion. A pooled sample was created from all samples and used for MS-MS runs using AquirX. For each AquirX run, a blank and the pool was run in MS1, then four runs of MS2 ID were performed. AquirX was run in pos and neg separately. Functional analysis of untargeted metabolome data was performed with the online tool MetaboAnalyst 5.0 (46) (https://www.metaboanalyst.ca/), using the Mummichog algorithm (positive mode; 3 ppm; p-value cutoff=0.001) and selecting the Mus musculus KEGG pathways containing at least 3 entries.

qPCR Analysis

Intestinal tissues were homogenized in 0.5 ml of TRIZOL buffer using the Retsch Mixer Mill MM400 with two zirconia beads at 25 frequency/sec for 10 min. Then, 100 μl chloroform was added for mixing and incubation at room temperature for 3 min. Samples were centrifuged at 12,000 g, 4C for 15 min, transferred 300 μl of the aqueous phase to RNase-free tubes in order to mix with 150 μl 100% ethanol, then isolated, and the total RNA eluted with the NucleoSpin RNA XS kit. The quality of extracted RNA was assessed by Agilent RNA chips. The RNA concentration was determined by a NanoDrop spectrophotometer. The cDNA was synthesized by iScript Reverse Transcription Supermix (Bio-Rad #1708841) using 1 μg total RNA as the template. qPCR was performed using PowerUp SYBR Green Master Mix (Thermo fisher #A25742) on the QuantStudio Dx Real-Time PCR Instrument. Three-step amplification was applied for each primer set. There were two technical replicates for each primer set. Data was analyzed with the delta-delta Ct method (47), using the mouse gapdh gene as the internal control.

For the assessment of bacterial abundance, genomic DNA was extracted from mouse feces using the ZymoBIOMICS DNA kit (Fisher Scientific #50-125-1501), then quantified with the Quantifluor ONE dsDNA System (Promega #E4870). DNA was diluted to 10-50 ng/μl and then used as the template for qPCR using the universal 16S rRNA primers or P. goldsteinii ASF519 specific primers. Data were analyzed with the delta-delta Ct method (47), using the universal 16S rRNA as a reference.

### Statistical Analysis

Mice at similar ages were randomly allocated into experimental groups. No statistical methods were used to predetermine sample size. Prism software (GraphPad, v8.4.3) was used for all statistical analysis, unless otherwise specified. Student t tests, non-parametric Mann-Whitney U tests, and two-way ANOVA with Tukey's multiple comparison adjustment was performed as indicated in the figure legends. For all statistical test, the following p values were used: ****p<0.0001, ***p<0.001, **p<0.01, *p<0.05 and not significant (ns). All graphs show the mean with sd or sem specified, with each dot representing a biological sample (unless otherwise specified). In the figure legends, n numbers refer to biological replicates, unless otherwise specified.

### Examples 10. Experiments with Spent Media

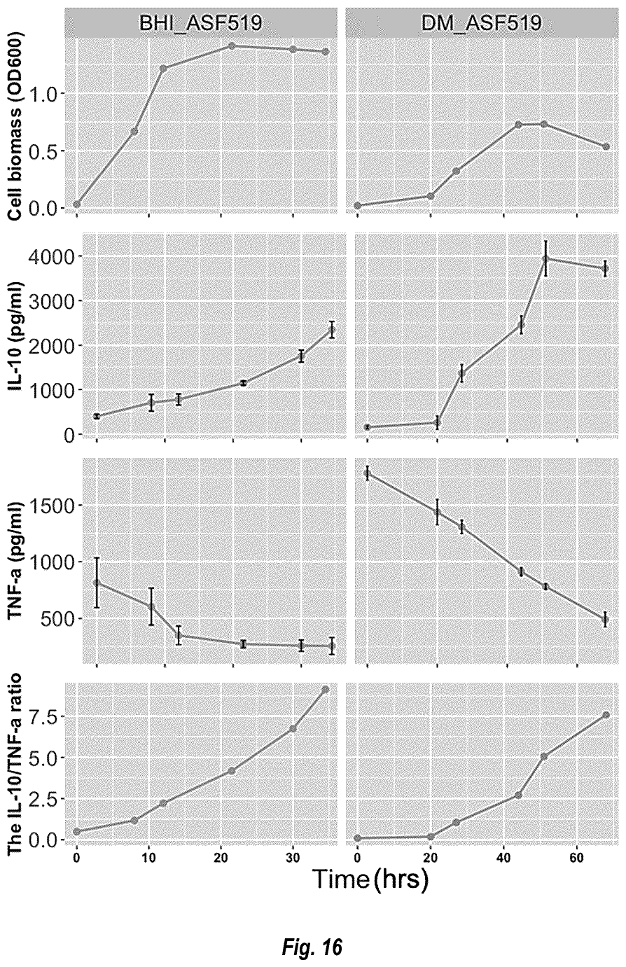

Experiments were performed to evaluate the in vitro effects of spent media of P. goldsteinii. Spent media were harvested at various time points during the growth of P. goldsteinii ASF519 in brain heart infusion broth (BHI) or defined medium (DM) under anaerobic condition, then filter-sterilized for in vitro stimulation of bone marrow derived dendritic cells (BMDCs). 1×10⁵ BMDCs in 140 μl were placed in a 96-well microplate, mixed with 60 μl of filter-sterilized spent media (SSMs) for 48-hour incubation in a CO₂ chamber, then supplemented with zymosan at the working concentration of 4 μg/ml for additional 16 hrs incubation under the same condition. Stimulated BMDC cultures were finally centrifuged at 500× g for 6 min to harvest supernatants for the measurement of IL-10 and TNF-α using ELISA kits. Data are shown in FIG. 16, wherein IL-10 levels of stimulated BMDCs was stimulated by spent media, while TNF-α levels were decreased. Since IL-10 has a role in controlling autoimmunity while TNF-α can promote autoimmunity, these effects could help to control symptoms of autoimmune disorders.

The impact on Treg proliferation was also tested by coculturing stimulated BMDCs with splenocytes. 1×10\hat{ }6 BMDCs in 1 ml placed in a 24-well plate were stimulated with 0.5 ml of SSMs collected at 23 hrs of ASF519 growth or 0.5 ml of BHI broth as the control. Then, cells were incubated at 37° C. in the CO₂ chamber for 2 days, followed by supplementation of zymosan at 4 μg/ml and additional 16 hours incubation under the same conditions. Fresh splenocytes from 129S1 were diluted to 1×10\hat{ }6 cells/ml with 1× PBS, stained with Celltrace violet at 10 μM, 37° C. for 20 min, washed once with 5V cRPMI, and finally resuspended in cRPMI at 2×10\hat{ }6 cell/ml. To coculture BMDCs and splenocytes, the supernatants were removed from stimulated BMDCs by centrifugation, cells were resuspended in 0.5 ml cRPMI, and then 0.5 ml of stained splenocytes were added for a 4-day incubation in a CO₂ incubator at 37° C. The Treg proliferation was analyzed by flowcytometry with mouse CD4 and FoxP3 antibodies.

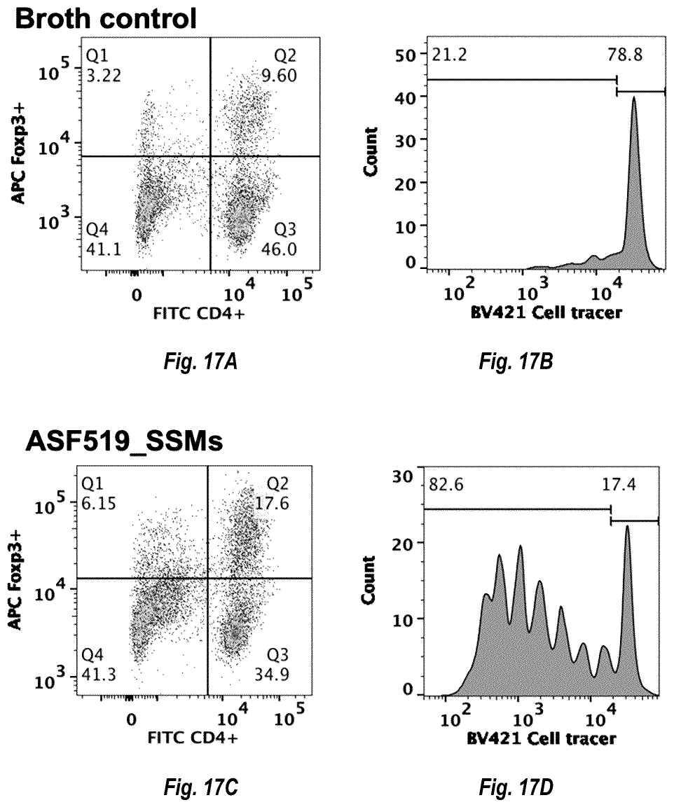

Results in FIGS. 17A-17D show that spent media of Parabacteroides goldsteinii promote CD4⁺FoxP3⁺ Treg expansion. An expansion of Tregs would be predicted to induce immune tolerance and reduce autoimmunity.

### REFERENCES

1. S. Kawamoto, M. Maruya, L. M. Kato, W. Suda, K. Atarashi, Y. Doi, Y. Tsutsui, H. Qin, K. Honda, T. Okada, M. Hattori, S. Fagarasan, Foxp3+ T Cells Regulate Immunoglobulin A Selection and Facilitate Diversification of Bacterial Species Responsible for Immune Homeostasis. Immunity. 41, 152-165 (2014).
2. J. L. Round, S. K. Mazmanian, The gut microbiota shapes intestinal immune responses during health and disease. Nat. Rev. Immunol. 9, 313-323 (2009).
3. D. Zhang, G. Chen, D. Manwani, A. Mortha, C. Xu, J. J. Faith, R. D. Burk, Y. Kunisaki, J.-E. Jang, C. Scheiermann, M. Merad, P. S. Frenette, Neutrophil ageing is regulated by the microbiome. Nature. 525, 528-532 (2015).
4. K. S. Josefsdottir, M. T. Baldridge, C. S. Kadmon, K. Y. King, Antibiotics impair murine hematopoiesis by depleting the intestinal microbiota. Blood. 129, 729-739 (2017).
5. M. Wymore Brand, M. J. Wannemuehler, G. J. Phillips, A. Proctor, A.-M. Overstreet, A. E. Jergens, R. P. Orcutt, J. G. Fox, The Altered Schaedler Flora: Continued Applications of a Defined Murine Microbial Community. ILAR J. 56, 169-178 (2015).
6. W. E. Ruff, T. M. Greiling, M. A. Kriegel, Host-microbiota interactions in immune-mediated diseases. Nat. Rev. Microbiol. (2020), doi:10.1038/s41579-020-0367-2.
7. T. S. Postler, S. Ghosh, Understanding the Holobiont: How Microbial Metabolites Affect Human Health and Shape the Immune System. Cell Metab. 26, 110-130 (2017).
8. J. Elzinga, J. van der Oost, W. M. de Vos, H. Smidt, The Use of Defined Microbial Communities To Model Host-Microbe Interactions in the Human Gut. Microbiol. Mol. Biol. Rev. 83 (2019), doi:10.1128/MMBR.00054-18.
9. K. Atarashi, T. Tanoue, K. Oshima, W. Suda, Y. Nagano, H. Nishikawa, S. Fukuda, T. Saito, S. Narushima, K. Hase, S. Kim, J. V. Fritz, P. Wilmes, S. Ueha, K. Matsushima, H. Ohno, B. Olle, S. Sakaguchi, T. Taniguchi, H. Morita, M. Hattori, K. Honda, Treg induction by a rationally selected mixture of Clostridia strains from the human microbiota. Nature. 500, 232-236 (2013).
10. T. Tanoue, S. Morita, D. R. Plichta, A. N. Skelly, W. Suda, Y. Sugiura, S. Narushima, H. Vlamakis, I. Motoo, K. Sugita, A. Shiota, K. Takeshita, K. Yasuma-Mitobe, D. Riethmacher, T. Kaisho, J. M. Norman, D. Mucida, M. Suematsu, T. Yaguchi, V. Bucci, T. Inoue, Y. Kawakami, B. Olle, B. Roberts, M. Hattori, R. J. Xavier, K. Atarashi, K. Honda, A defined commensal consortium elicits CD8 T cells and anti-cancer immunity. Nature. 565, 600-605 (2019).
11. N. Geva-Zatorsky, E. Sefik, L. Kua, L. Pasman, T. G. Tan, A. Ortiz-Lopez, T. B. Yanortsang, L. Yang, R. Jupp, D. Mathis, C. Benoist, D. L. Kasper, Mining the Human Gut Microbiota for Immunomodulatory Organisms. Cell. 168, 928-943.e11 (2017).
12. M. B. Biggs, G. L. Medlock, T. J. Moutinho, H. J. Lees, J. R. Swann, G. L. Kolling, J. A. Papin, Systems-level metabolism of the altered Schaedler flora, a complete gut microbiota. ISME J. 11, 426-438 (2017).
13. J. Collins, R. Borojevic, E. F. Verdu, J. D. Huizinga, E. M. Ratcliffe, Intestinal microbiota influence the early postnatal development of the enteric nervous system. Neurogastroenterol. Motil. 26, 98-107 (2014).
14. I. I. Ivanov, R. de L. Frutos, N. Manel, K. Yoshinaga, D. B. Rifkin, R. B. Sartor, B. B. Finlay, D. R. Littman, Specific microbiota direct the differentiation of IL-17-producing T-helper cells in the mucosa of the small intestine. Cell Host Microbe. 4, 337-349 (2008).
15. M. B. Geuking, J. Cahenzli, M. A. E. Lawson, D. C. K. Ng, E. Slack, S. Hapfelmeier, K. D. McCoy, A. J. Macpherson, Intestinal bacterial colonization induces mutualistic regulatory T cell responses. Immunity. 34, 794-806 (2011).
16. I. Mosconi, M. B. Geuking, M. M. Zaiss, J. C. Massacand, C. Aschwanden, C. K. C. Kwong Chung, K. D. McCoy, N. L. Harris, Intestinal bacteria induce TSLP to promote mutualistic T-cell responses. Mucosal Immunol. 6, 1157-1167 (2013).
17. S. Hapfelmeier, M. A. E. Lawson, E. Slack, J. K. Kirundi, M. Stoel, M. Heikenwalder, J. Cahenzli, Y. Velykoredko, M. L. Balmer, K. Endt, M. B. Geuking, R. Curtiss 3rd, K. D. McCoy, A. J. Macpherson, Reversible microbial colonization of germ-free mice reveals the dynamics of IgA immune responses. Science. 328, 1705-1709 (2010).
18. D. Bouskra, C. Brezillon, M. Bérard, C. Werts, R. Varona, I. G. Boneca, G. Eberl, Lymphoid tissue genesis induced by commensals through NOD1 regulates intestinal homeostasis. Nature. 456, 507-510 (2008).
19. S. A. Keilbaugh, M. E. Shin, R. F. Banchereau, L. D. McVay, N. Boyko, D. Artis, J. J. Cebra, G. D. Wu, Activation of RegIIIbeta/gamma and interferon gamma expression in the intestinal tract of SCID mice: an innate response to bacterial colonisation of the gut. Gut. 54, 623-629 (2005).
20. S. Lavoie, K. L. Conway, K. G. Lassen, H. B. Jijon, H. Pan, E. Chun, M. Michaud, J. K. Lang, C. A. Gallini Comeau, J. M. Dreyfuss, J. N. Glickman, H. Vlamakis, A. Ananthakrishnan, A. Kostic, W. S. Garrett, R. J. Xavier, The Crohn's disease polymorphism, ATG16L1 T300A, alters the gut microbiota and enhances the local Th1/Th17 response. Elife. 8 (2019), doi:10.7554/eLife.39982.
21. X. Li, M. L. Ellis, A. E. Dowell, R. Kumar, C. D. Morrow, T. R. Schoeb, J. Knight, Response of germ-free mice to colonization with O. formigenes and altered Schaedler flora. Appl. Environ. Microbiol. 82, 6952-6960 (2016).
22. M. P. Burrows, P. Volchkov, K. S. Kobayashi, A. V. Chervonsky, Microbiota regulates type 1 diabetes through Toll-like receptors. Proc. Natl. Acad. Sci. U. S. A. 112, 9973-9977 (2015).
23. T. N. Ellis, S. A. Leiman, M. J. Kuehn, Naturally produced outer membrane vesicles from Pseudomonas aeruginosa elicit a potent innate immune response via combined sensing of both lipopolysaccharide and protein components. Infect. Immun. 78, 3822-3831 (2010).
24. R. C. Alaniz, B. L. Deatherage, J. C. Lara, B. T. Cookson, Membrane vesicles are immunogenic facsimiles of Salmonella typhimurium that potently activate dendritic cells, prime B and T cell responses, and stimulate protective immunity in vivo. The Journal of Immunology. 180 (2008), pp. 3612.3-3612.
25. Ismail Salim, Hampton Mark B., Keenan Jacqueline I., Helicobacter pylori Outer Membrane Vesicles Modulate Proliferation and Interleukin-8 Production by Gastric Epithelial Cells. Infect. Immun. 71, 5670-5675 (2003).
26. O. Y. Kim, H. T. Park, N. T. H. Dinh, S. J. Choi, J. Lee, J. H. Kim, S.-W. Lee, Y. S. Gho, Bacterial outer membrane vesicles suppress tumor by interferon-y-mediated antitumor response. Nat. Commun. 8, 626 (2017).
27. G. Haskó, J. Linden, B. Cronstein, P. Pacher, Adenosine receptors: therapeutic aspects for inflammatory and immune diseases. Nat. Rev. Drug Discov. 7, 759-770 (2008).
28. B. B. Fredholm, Adenosine, an endogenous distress signal, modulates tissue damage and repair. Cell Death Differ. 14, 1315-1323 (2007).
29. Z. Deng, Z. He, G. Maksaev, R. M. Bitter, M. Rau, J. A. J. Fitzpatrick, P. Yuan, Cryo-EM structures of the ATP release channel pannexin 1. Nat. Struct. Mol. Biol. 27, 373-381 (2020).
30. S. Turley, L. Poirot, M. Hattori, C. Benoist, D. Mathis, Physiological β Cell Death Triggers Priming of Self-reactive T Cells by Dendritic Cells in a Type-1 Diabetes Model. J. Exp. Med. 198, 1527-1537 (2003).
31. Y. Shen, M. L. Giardino Torchia, G. W. Lawson, C. L. Karp, J. D. Ashwell, S. K. Mazmanian, Outer membrane vesicles of a human commensal mediate immune regulation and disease protection. Cell Host Microbe. 12, 509-520 (2012).
32. S. K. Mazmanian, J. L. Round, D. L. Kasper, A microbial symbiosis factor prevents intestinal inflammatory disease. Nature. 453, 620-625 (2008).
33. H. Chu, A. Khosravi, I. P. Kusumawardhani, A. H. K. Kwon, A. C. Vasconcelos, L. D. Cunha, A. E. Mayer, Y. Shen, W.-L. Wu, A. Kambal, S. R. Targan, R. J. Xavier, P. B. Ernst, D. R. Green, D. P. B. McGovern, H. W. Virgin, S. K. Mazmanian, Gene-microbiota interactions contribute to the pathogenesis of inflammatory bowel disease. Science. 352, 1116-1120 (2016).
34. H. Chen, P.-K. Nwe, Y. Yang, C. E. Rosen, A. A. Bielecka, M. Kuchroo, G. W. Cline, A. C. Kruse, A. M. Ring, J. M. Crawford, N. W. Palm, A Forward Chemical Genetic Screen Reveals Gut Microbiota Metabolites That Modulate Host Physiology. Cell. 177, 1217-1231.e18 (2019).
35. L. J. Cohen, H.-S. Kang, J. Chu, Y.-H. Huang, E. A. Gordon, B. V. B. Reddy, M. A. Ternei, J. W. Craig, S. F. Brady, Functional metagenomic discovery of bacterial effectors in the human microbiome and isolation of commendamide, a GPCR G2A/132 agonist. Proceedings of the National Academy of Sciences. 112 (2015), pp. E4825-E4834.
36. T. Gnad, S. Scheibler, I. von Kügelgen, C. Scheele, A. Kilić, A. Glöde, L. S. Hoffmann, L. Reverte-Salisa, P. Horn, S. Mutlu, A. El-Tayeb, M. Kranz, W. Deuther-Conrad, P. Brust, M. E. Lidell, M. J. Betz, S. Enerbäck, J. Schrader, G. G. Yegutkin, C. E. Müller, A. Pfeifer, Adenosine activates brown adipose tissue and recruits beige adipocytes via A2A receptors. Nature. 516, 395-399 (2014).
37. N. Schulz, K.-C. Liu, J. Charbord, C. L. Mattsson, L. Tao, D. Tworus, O. Andersson, Critical role for adenosine receptor A2a in β-cell proliferation. Mol Metab. 5, 1138-1146 (2016).
38. J. A. Pearson, F. S. Wong, L. Wen, The importance of the Non Obese Diabetic (NOD) mouse model in autoimmune diabetes. J. Autoimmun. 66, 76-88 (2016).
39. A. L. O'Kell, C. Wasserfall, B. Catchpole, L. J. Davison, R. S. Hess, J. A. Kushner, M. A. Atkinson, Comparative Pathogenesis of Autoimmune Diabetes in Humans, NOD Mice, and Canines: Has a Valuable Animal Model of Type 1 Diabetes Been Overlooked? Diabetes. 66, 1443-1452 (2017).
40. D. D. Brand, K. A. Latham, E. F. Rosloniec, Collagen-induced arthritis. Nat. Protoc. 2, 1269-1275 (2007).
41. V. Trouplin, N. Boucherit, L. Gorvel, F. Conti, G. Mottola, E. Ghigo, Bone marrow-derived macrophage production. J. Vis. Exp. (2013) (available at https://www.ncbi.nlm.nih.gov/pmc/articles/pmc3991821/).
42. S. Picelli, O. R. Faridani, A. K. Björklund, G. Winberg, S. Sagasser, R. Sandberg, Full-length RNA-seq from single cells using Smart-seq2. Nat. Protoc. 9, 171-181 (2014).
43. D. T. Truong, E. A. Franzosa, T. L. Tickle, M. Scholz, G. Weingart, E. Pasolli, A. Tett, C. Huttenhower, N. Segata, MetaPhlAn2 for enhanced metagenomic taxonomic profiling. Nat. Methods. 12, 902-903 (2015).
44. E. Pasolli, L. Schiffer, P. Manghi, A. Renson, V. Obenchain, D. T. Truong, F. Beghini, F. Malik, M. Ramos, J. B. Dowd, C. Huttenhower, M. Morgan, N. Segata, L. Waldron, Accessible, curated metagenomic data through ExperimentHub. Nat. Methods. 14, 1023-1024 (2017).
45. B. Scallon, A. Cai, N. Solowski, A. Rosenberg, X.-Y. Song, D. Shealy, C. Wagner, Binding and functional comparisons of two types of tumor necrosis factor antagonists. J. Pharmacol. Exp. Ther. 301, 418-426 (2002).
46. J. Chong, O. Soufan, C. Li, I. Caraus, S. Li, G. Bourque, D. S. Wishart, J. Xia, MetaboAnalyst 4.0: towards more transparent and integrative metabolomics analysis. Nucleic Acids Res. 46, W486-W494 (2018).
47. K. J. Livak, T. D. Schmittgen, Analysis of Relative Gene Expression Data Using Real-Time Quantitative PCR and the 2-ΔΔCT Method. Methods. 25 (4), 402-8 (2001).

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A composition comprising isolated extracellular vesicles prepared from a sample comprising Parabacteroides goldsteinii, wherein bacteria have been removed from the composition.

2. (canceled)

3. The composition of claim 1, wherein bacteria have been removed by filtration.

4. The composition of claim 1, wherein the extracellular vesicles:
a. comprise proteins, nucleic acids, lipids, metabolites, and/or outer membrane of Parabacteroides goldsteinii; and/or
b. are prepared using gel filtration, optionally followed by gradient ultracentrifugation.

5. (canceled)

6. A method of promoting immune tolerance comprising administering the composition of claim 1 to a cell or comprising administering live Parabacteroides goldsteinii to a cell, wherein the immune tolerance of the cell is characterized by any one or more of:
i. increased IL-10 and/or adenosine production;
ii. activation of adenosine receptor A2a;
iii. increased expression of Il10, lipocalin-2 (Lcn2), and/or indoleamine 2, 3-dioxygenases (Ido1/2);
iv. reduced expression of Cd40, Il12b, Il23a, Il1f9, and/or Il-36 gamma;
v. increased autophagy; and
vi. reduced TNF-α, IL-4, and/or IL-5 production.

7. The method of claim 6, wherein the increased IL-10 production is stimulated all or partly through the pannexin1-CD73-A2a axis.

8. The method of claim 6, wherein the cell is a macrophage, T cell, or dendritic cell.

9. (canceled)

10. The method of claim 6, wherein the immune tolerance is characterized by increased adenosine production and/or activation of adenosine receptor A2a of the dendritic cell.

11. The method of claim 6, wherein the cell is comprised in a cell culture and the administering is in vitro.

12. The method of claim 6, wherein the cell is comprised in a subject and the administering is oral delivery of the composition.

13. The method of claim 12, wherein the administering does not change the composition of the subject's microbiome.

14. A method of preventing or treating an autoimmune or inflammatory disease comprising administering live Parabacteroides goldsteinii and/or the composition of claim 1 to a subject, optionally wherein the administering produces:
a. increased IL-10 and/or adenosine production;
b. activation of adenosine receptor A2a;
c. increased autophagy; and/or
d. reduced TNF-α, IL-4, and/or IL-5 production, in one or more cells of the subject.

15. The method of claim 14, wherein the administering comprises administering live Parabacteroides goldsteinii to a subject and the administering changes the composition of the subject's microbiome.

16. The method of claim 14, wherein the administering does not change the composition of the subject's microbiome.

17. The method of claim 14, wherein the administering is by oral administration.

18. A method of preventing or treating an autoimmune or inflammatory disease comprising administering an A2a adenosine receptor agonist to a subject, optionally wherein the administering produces:
a. increased IL-10 production;
b. increased autophagy; and/or
c. reduced TNF-α, IL-4, and/or IL-5 production, in one or more cells of the subject.

19. A method of preventing or treating an autoimmune or inflammatory disease comprising administering a composition comprising (1) live Parabacteroides goldsteinii and/or the composition of claim 1 and (2) an A2a adenosine receptor agonist to a subject, optionally wherein the administering produces:
a. increased IL-10 and/or adenosine production;
b. activation of adenosine receptor A2a;
c. increased autophagy; and/or
d. reduced TNF-α, IL-4, and/or IL-5 production, in one or more cells of the subject.

20. The method of claim 18, wherein:
a. the A2a adenosine receptor agonist is adenosine, regadenoson, spongosine, sonedenoson (MRE-0094), apadenoson (ATL-146e, BMS 068645), evodenoson, ATL-313, DE-112, UK-371104 27, UK-432097, or GW328267X; and/or
b. the administering increases levels of adenosine and/or IL10 in the subject, optionally wherein the increased levels of adenosine and/or IL10 are measured in the gut or synovial tissue or fluid.

21. (canceled)

22. (canceled)

23. The method of claim 14, wherein:
a. the administering decreases levels of TNF-α, IL-4, and/or IL-5 in the subject, optionally wherein the decreased levels of TNF-α, IL-4, and/or IL-5 are measured in the gut or synovial tissue or fluid; and/or
b. the autoimmune or inflammatory disease is rheumatoid arthritis (RA).

24. (canceled)

25. (Canceled)

26. A method of producing extracellular vesicles from Parabacteroides goldsteinii comprising:
a. growing Parabacteroides goldsteinii in culture;
b. collecting a sample comprising Parabacteroides goldsteinii cells and cell culture supernatant;
c. removing Parabacteroides goldsteinii cells from the sample;
d. performing gel filtration and gradient ultracentrifugation on the sample, wherein the gel filtration and gradient ultracentrifugation are separate steps performed in either order; and
e. isolating extracellular vesicles.

27. A method of preventing or treating an autoimmune or inflammatory disease comprising administering the extracellular vesicles of claim 26 to a subject, optionally wherein the administering produces:
a. increased IL-10 and/or adenosine production;
b. activation of adenosine receptor A2a;
c. increased autophagy; and/or
d. reduced TNF-α, IL-4, and/or IL-5 production, in one or more cells of the subject.
